(19) United States
(12) Patent Application Publication
(10) Pub. No.: US 2021/0338577 A1
(43) Pub. Date: Nov. 4, 2021

(54) BIO-INSPIRED DEGRADABLE TOUGH ADHESIVES FOR DIVERSE WET SURFACES

(71) Applicant: President and Fellows of Harvard College, Cambridge, MA (US)

(72) Inventors: Benjamin Ross Freedman, Brookline, MA (US); Oktay R. Uzun, Boston, MA (US); David J. Mooney, Sudbury, MA (US); Nadja M. Maldonado Luna, Cambridge, MA (US)

(21) Appl. No.: 17/283,412

(22) PCT Filed: Oct. 11, 2019
(86) PCT No.: PCT/US2019/055779

§ 371 Date: Apr. 7, 2021

Related U.S. Application Data
(60) Provisional application No. 62/744,756, filed on Oct. 12, 2018.

(51) Int. Cl. A61K 9/06; A61K 47/69; A61K 47/34; A61K 47/32; A61K 47/36; A61L 24/00; A61L 24/04

(52) U.S. Cl. CPC A61K 9/06; A61K 47/6903; A61K 47/34; A61L 24/043; A61K 47/36; A61L 24/0031; A61K 47/32

(57) ABSTRACT

The present invention is directed to a biodegradable tough adhesive material comprising an interpenetrating networks (IPN) hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks; a high density primary amine polymer; and a coupling agent. The present invention also provides methods preparing and using the biodegradable tough adhesive material.

### CROSS-REFERENCE TO RELATED APPLICATION

The instant application claims priority to U.S. Provisional Application No. 62/744,756, filed on Oct. 12, 2018, the entire contents of which are incorporated herein by reference.

### GOVERNMENT SUPPORT

The invention was made with U.S. government support under AG057135 awarded by the National Institutes of Health. The government has certain rights in the invention.

### BACKGROUND OF THE INVENTION

Hydrogels are crosslinked hydrophilic polymer structures that hold many biomedical and pharmaceutical applications. They can be used as scaffolds for tissue engineering, vehicles for drug delivery, coatings for medical devices, wound dressings, among others. Recent research has developed hydrogels with fracture energies several times greater than native tissues (termed “tough gels”). These materials are formed from an interpenetrating network (IPN) of alginate and polyacrylamide. Traditional hydrogels tend to be stiff and brittle, however, these tough gels demonstrate exceptional mechanical properties, being able to stretch up to 20× their initial length without rupture. Furthermore, studies on the biocompatibility of alginate and polyacrylamide tough gels have shown promise in vitro and in vivo, rendering these tough gels suitable for use as a potential biomaterial. When combined with an adhesive bridging polymer, these tough gels are able to achieve strong adhesion to wet and dynamically moving tissue surfaces. Although these tough gels have achieved exceptionally high fracture energies, adhesion to wet tissue surfaces and excellent biocampatibility, there remains an unmet need for tunable degradation of these tough gels for enabling their use in various medical treatments, for example, in biosurgery applications.

Therefore, there remains an unmet need for tissue adhesives that exhibit strong bonding to the desired surface in particular wet surfaces of biological tissues, can withstand significant mechanical stresses and strains, and are biodegradable.

### SUMMARY OF THE INVENTION

The compositions and methods disclosed in the present invention are based, at least in part, on the development of degradable tough gels and tough adhesive materials using biocompatible, biodegradable covalent crosslinkers. In particular, the present inventors have synthesized degradable and tough hydrogels using different biodegradable covalent crosslinkers to achieve high fracture toughness. These tough hydrogels and tough adhesive materials may be engineered to have tunable degradation properties by adjusting the concentration and composition of the covalent crosslinker, permitting degradation of the material to occur naturally for their use in various biomedical applications, e.g., in the development of biosurgery products to prevent excessive blood loss and provide wound sealing.

Furthermore, the biodegradable tough adhesive materials disclosed in the present invention lead to extremely high fracture energy (e.g., about 10 kJ/m² to about 20 kJ/m²), which is higher than native cartilage. Adhesion is fast (within minutes), independent of blood exposure, and compatible with in vivo dynamic movements (e.g., the beating heart). The biodegradable adhesive materials can be in the form of preformed patches or injectable gels that can be in situ adhered on the target surface (e.g., can act as a surgical glue providing a suture-less adhesive).

Accordingly, in one aspect, the present invention provides a composition comprising a biodegradable interpenetrating networks (IPN) hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks.

In another aspect, the present invention provides a composition comprising a biodegradable tough adhesive material, comprising a) an IPN hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks; b) an adhesive bridging polymer; and c) a coupling agent.

In some embodiments, the first polymer is selected from the group consisting of polyacrylamide, poly(vinyl alcohol) (PVA), polyethylene glycol (PEG), polyphosphazene, collagen, gelatin, poly(acrylate), poly(methacrylate), poly(methacrylamide), poly(acrylic acid), poly(N-isopropylacrylamide), poly(N,N-dimentylacrylamide), poly(allylamine) and copolymers thereof. In a particular embodiment, the first polymer network is polyacrylamide.

In some embodiments, the first polymer is selected from the group consisting of polyacrylamide, poly(hydroxyethylmethacrylate) (PHEMA), poly(vinyl alcohol) (PVA), polyethylene glycol (PEG), polyphosphazene, collagen, gelatin, poly(acrylate), poly(methacrylate), poly(methacrylamide), poly(acrylic acid), poly(N-isopropylacrylamide) (PNIPAM), poly(N,N-dimentylacrylamide), poly(allylamine) and copolymers thereof. In a particular embodiment, the first polymer network is polyacrylamide, which can form a covalently cross-linked polymeric network via free-radical polymerization, click chemistry, etc.

In some embodiments, the biodegradable covalent crosslinker is selected from the group consisting of a poly(ethylene glycol) acrylate, a gelatin acrylate, a hyaluronic acid acrylate, an alginate acrylate a poloxamer acrylate, and a disulfide-based crosslinker. In some embodiments, the biodegradable covalent crosslinker is selected from the group consisting of a poly(ethylene glycol) acrylate, a gelatin acrylate, a hyaluronic acid acrylate and an alginate acrylate. In some embodiments, the biodegradable covalent crosslinker is selected from the group consisting of a poly(ethylene glycol) diacrylate (PEGDA), gelatin methacrylate (GeIMA), alginate methacrylate (AlgMA), hyaluronic acid methacrylate (HAMA), a poloxamer acrylate, a disulfide-based acrylate, and N,N′-bis(acryloyl)cystamine (Cys). In some embodiments, the biodegradable covalent crosslinker is selected from the group consisting of poly(ethylene glycol) diacrylate 250 (PEGDA 250), gelatin methacrylate (GeIMA), hyaluronic acid methacrylate (HAMA), oxidized alginate methacrylate (OxAlgMA), poloxamer diacrylate (Polox DA), bis(2-methacryloyl)oxyethyl disulfide (Bis), and N,N′-bis(acryloyl)cystamine (Cys). In some embodiments, the biodegradable covalent crosslinker is selected from the group consisting of a poly(ethylene glycol) diacrylate (PEGDA), a gelatin methacrylate (GelMA), a methacrylated alginate (AlgMA).

In some embodiments, the biodegradable covalent crosslinker has a molecular weight of about 100 Da to about 40,000 Da. In an embodiment, the biodegradable covalent crosslinker has a molecular weight of about 250 Da to about 20,000 Da. In some additional embodiments, the biodegradable covalent crosslinker is a PEGDA having a molecular weight of about 250 Da, about 10,000 Da, or about 20,000 Da. In an embodiment, the biodegradable covalent crosslinker is GelMA. In another embodiment, the biodegradable covalent crosslinker is AlgMA-5 Mrad (irradiated alginate to create low molecular weight). In yet another embodiment, the biodegradable covalent crosslinker is a PEGDA having a molecular weight of about 10,000 Da (PEGDA 10 k). In a particular embodiment, the biodegradable covalent crosslinker is a PEGDA having a molecular weight of about 250 Da (PEGDA 250).

In an embodiment, the concentration of the poly(ethylene glycol) diacrylate (PEGDA), such as PEGDA 250, in the hydrogel is about 0.001 wt. % to 0.05 wt. % based on the weight of the first polymer, e.g., about 0.005 wt. % to 0.02 wt. % based on the weight of the first polymer. In another embodiment, the concentration of PEGDA 250 in the hydrogel is about 0.0015 wt. % to 0.06 wt. % based on the weight of the polyacrylamide.

In another embodiment, the concentration of PEGDA 10 k in the hydrogel is about 0.003 wt. % to 0.06 wt. % based on the weight of the polyacrylamide.

In one embodiment, the concentration of the poloxamer acrylate, such as poloxamer diacrylate (Polox DA), in the hydrogel is about 0.001 wt. % to 0.05 wt. % based on the weight of the first polymer, e.g., about 0.01 wt. % to 0.025 wt. % based on the weight of the first polymer.

In yet another embodiment, the concentration of gelatin methacrylate (GeIMA) in the hydrogel is about 0.012 wt. % to 0.2 wt. % based on the weight of the polyacrylamide. In one embodiment, the concentration of the gelatin methacrylate (GeIMA) in the hydrogel is about 0.001 wt. % to 0.05 wt. % based on the weight of the first polymer, e.g., about 0.003 wt. % to 0.01 wt. % based on the weight of the first polymer.

In an embodiment, the concentration of AlgMA-5 Mrad in the hydrogel is about 0.012 wt. % to 0.2 wt. % based on the weight of the polyacrylamide.

In one embodiment, the concentration of the oxidized alginate methacrylate (OxAlgMA) in the hydrogel is about 0.001 wt. % to 0.05 wt. % based on the weight of the first polymer, e.g., about 0.005 wt. % to 0.02 wt. % based on the weight of the first polymer.

In one embodiment, the concentration of the hyaluronic acid methacrylate (HAMA) in the hydrogel is about 0.001 wt. % to 0.05 wt. % based on the weight of the first polymer, e.g., about 0.005 wt. % to 0.02 wt. % based on the weight of the first polymer.

In one embodiment, the concentration of the disulfide-based acrylate in the hydrogel is about 0.005 wt. % to 0.03 wt. % based on the weight of the first polymer, e.g., about 0.01 wt. % to 0.025 wt. % based on the weight of the first polymer.

In one embodiment, the concentration of N,N′-bis(acryloyl)cystamine (Cys) in the hydrogel is about 0.0005 wt. % to 0.01 wt. % based on the weight of the first polymer, e.g., about 0.001 wt. % to 0.002 wt. % based on the weight of the first polymer.

In some embodiments, the second polymer is selected from the group consisting of alginate, pectate, carboxymethyl cellulose, oxidized carboxymethyl cellulose, hyaluronate, chitosan, κ-carrageenan, ι-carrageenan and λ-carrageenan, wherein the alginate, carboxymethyl cellulose, hyaluronate chitosan, κ-carrageenan, ι-carrageenan and λ-carrageenan are each optionally oxidized, wherein the alginate, carboxymethyl cellulose, hyaluronate chitosan, κ-carrageenan, ι-carrageenan and λ-carrageenan optionally include one or more groups selected from the group consisting of methacrylate, acrylate, acrylamide, methacrylamide, thiol, hydrazine, tetrazine, norbornene, transcyclooctene and cyclooctyne. In a particular embodiment, the second polymer network comprises alginate. In one embodiment, the alginate is modified alginate or oxidized alginate. Modified alginates, such as but not limited to the modified alginates, functionalized alginates, oxidized alginates (including partially oxidized alginates), and oxidized/reduced alginates described in International Patent Application Publication Nos. WO 2015/154082, WO 2017/075055, the entire contents of which are both incorporated herein by reference*.

In some embodiments, the alginate is comprised of a mixture of a high molecular weight alginate and a low molecular weight alginate. In certain embodiments, the ratio of the high molecular weight alginate to the low molecular weight alginate is between 0% and 100%, e.g., between 10-90%, 10-80%, 10-70%, 10-60%, 10-50%, 10-40%, 10-30%, 10-20%, 20-90%, 20-80%, 20-70%, 20-60%, 20-50%, 20-40%, 20-30%, 30-70%, 30-60%, 30-50%, 30-40%, 40-60%, 60-40%. In a particular embodiment, the ratio of the high molecular weight alginate to the low molecular weight alginate is about 50%.

In some embodiments, the crosslinking agents that promote ionic crosslinks include CaCl₂, CaSO₄, CaCO₃, hyaluronic acid, and polylysine.

In some embodiments, the hydrogel comprises about 30% to about 98% water.

In some embodiments, the hydrogel is fabricated in the form of a patch.

In some embodiments, the first network and the second network are covalently coupled. The nature of the bonds between first and second networks is determined using Fourier Transform Infrared (FTIR) spectra or Thermogravimetric analysis (TGA). The biodegradable interpenetrating network hydrogel comprises enhanced mechanical properties selected from the group consisting of self-healing ability, increased fracture toughness, increased ultimate tensile strength, and increased rupture stretch. In some embodiments, the hydrogels have a fracture energy between about 2.5 kJ/m² to about 20 kJ/m². In a particular embodiment, the hydrogel has a fracture energy of about 20 kJ/m².

In some embodiments, the hydrogel is hydrolytically degradable. In some additional embodiments, the hydrogel is enzymatically degradable.

In some embodiments, the adhesive bridging polymer is a high density primary amine polymer. In some embodiments, the high density primary amine polymer comprises at least one primary amine per monomer unit. In certain embodiments, the high density primary amine polymer is selected from the group consisting of chitosan, gelatin, collagen, polyallylamine, polylysine, and polyethylenimine. In a particular embodiment, the high density primary amine polymer is chitosan.

In some embodiments, the coupling agent includes a first carboxyl activating agent. In certain embodiments, the first carboxyl activating agent is a carbodiimide. In some embodiments, the carbodiimide is selected from the group consisting of 1-ethyl-3-(3-dimethylaminopropyl)carbodiimide (EDC, EDAC or EDCI), dicyclohexylcarbodiimide (DCC) and diisopropylcarbodiimide (DIC). In some embodiments, the coupling agent further includes a second carboxyl activating agent. In certain embodiments, the second carboxyl activating agent is N-hydroxysuccinimide (NHS), N-hydroxysulfosuccinimide (sulfo-NHS), hydroxybenzotriazole (HOBt), dimethylaminopyridine (DMAP), Hydroxy-3,4-dihydro-4-oxo-1,2,3-benzotriazine (HOOBt/HODhbt), 1-Hydroxy-7-aza-1H-benzotriazole (HOAt), Ethyl 2-cyano-2-(hydroximino)acetate, Benzotriazol-1-yloxy-tris(dimethylamino)-phosphonium hexafluorophosphate (BOP), Benzotriazol-1-yloxy-tripyrrolidino-phosphonium hexafluorophosphate, 7-Aza-benzotriazol-1-yloxy-tripyrrolidinophosphonium hexafluorophosphate), Ethyl cyano(hydroxyimino)acetato-O²)-tri-(1-pyrrolidinyl)-phosphonium hexafluorophosphate, 3-(Diethoxy-phosphoryloxy)-1,2,3-benzo[d] triazin-4(3H)-one, 2-(1H-Benzotriazol-1-yl)-N,N,N′,N′-tetramethylaminium tetrafluoroborate/hexafluorophosphate, 2-(6-Chloro-1H-benzotriazol-1-yl)-N,N,N′,N′-tetramethylaminium hexafluorophosphate), N-[(5-Chloro-1H-benzotriazol-1-yl)-dimethylamino-morpholino]-uronium hexafluorophosphate N-oxide, 2-(7-Aza-1H-benzotriazol-1-yl)-N,N,N′,N′-tetramethylaminium hexafluorophosphate, 1-[1-(Cyano-2-ethoxy-2-oxoethylideneaminooxy)-dimethylamino-morpholino]-uronium hexafluorophosphate, 2-(1-Oxy-pyridin-2-yl)-1,1,3,3-tetramethylisothiouronium tetrafluoroborate, Tetramethylfluoroformamidinium hexafluorophosphate, N-Ethoxycarbonyl-2-ethoxy-1,2-dihydroquinoline, 2-Propanephosphonic acid anhydride, 4-(4,6-Dimethoxy-1,3,5-triazin-2-yl)-4-methylmorpholinium salts, (bis-Trichloromethylcarbonate, 1,1′-Carbonyldiimidazole.

In some embodiments, the high density primary amine polymer and the coupling agent are packaged separately. In certain embodiments, the high density primary amine polymer is in a solution and the coupling agent is in solid form. In some embodiments, the coupling agent is added to the high density primary amine polymer solution. In some embodiments, the concentration of the high density primary amine polymer in the solution is about 0.1% to about 50%. In certain embodiments, the coupling agent includes at least a first carboxyl activating agent and optionally a second carboxyl activating agent, and wherein the concentration of the first carboxyl activating agent in the solution is about 3 mg/ml to about 50 mg/ml. In some embodiments, the high density primary amine polymer is in a solution, the coupling agent is added to the high density primary amine polymer solution, and the solution is applied to the hydrogel.

In an aspect, the invention provides a composition comprising a biodegradable IPN hydrogel comprising a first polymer network and a second polymer network, wherein the first polymer network comprises polyacrylamide and a biodegradable covalent crosslinker, and the second polymer network comprises an alginate polymer.

In another aspect, the invention discloses a composition comprising a biodegradable adhesive material comprising: (a) a biodegradable interpenetrating networks hydrogel comprising a first polymer network and a second polymer network, wherein the first polymer network comprises polyacrylamide and a biodegradable covalent crosslinker, and the second polymer network comprises an alginate polymer; (b) an adhesive bridging polymer comprising chitosan; and (c) a coupling agent comprising EDC and sulfated NHS.

In one embodiment, the biodegradable covalent crosslinker is poly(ethylene glycol) diacrylate (PEGDA 250), poloxamer diacrylate (Polox DA), gelatin methacrylate (GelMA), oxidized alginate methacrylate (OxAlgMA), hyaluronic acid methacrylate (HAMA), bis(2-methacryloyl)oxyethyl disulfide (Bis), or N,N′-bis(acryloyl)cystamine (Cys).

In some preferred embodiments, the first polymer network and the second polymer network are covalently coupled.

In an aspect, the invention discloses method of making a biodegradable IPN hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks, the method comprising mixing a first polymer and a second polymer; and contacting the mixture with a biodegradable covalent crosslinker and an ionic crosslinker thereby making an IPN hydrogel.

In some embodiments, the biodegradable covalent crosslinker is poly(ethylene glycol) diacrylate (PEGDA 250), poloxamer diacrylate (Polox DA), gelatin methacrylate (GelMA), oxidized alginate methacrylate (OxAlgMA), hyaluronic acid methacrylate (HAMA), bis(2-methacryloyl)oxyethyl disulfide (Bis), or N,N′-bis(acryloyl)cystamine (Cys), and the ionic crosslinker comprises CaSO₄-2H₂O (calcium dihydrate). In a preferred embodiment, the first polymer network and the second polymer network are covalently coupled.

In some embodiments, the ratio between CaSO₄*2H₂O and the second polymer is between about 3.32 wt. % and 53.15 wt. %. In some embodiments, the first polymer is an acrylamide polymer and the second polymer is alginate, and wherein the polymer ratio between the polyacrylamide polymer and the alginate polymer is between about 66.67 wt. % and 94.12 wt. %, about 88.89 wt. % or about 85.71 wt. %.

In another aspect, the present invention provides a method of adhering a biodegradable IPN hydrogel to a surface (for example, a tissue), the method including the steps of: (a) applying a solution comprising a high density primary amine polymer and a coupling agent to the hydrogel; and (b) placing the hydrogel on the surface; wherein the hydrogel comprises a first polymer network and a second polymer network, wherein the first polymer network comprises a first polymer covalently crosslinked with a biodegradable covalent crosslinker and the second polymer network comprises a second polymer crosslinked with ionic or physical crosslinks.

In certain embodiments, the surface is tissue. In certain embodiments, the tissue is selected from the group consisting of heart tissue, skin tissue, blood vessel tissue, bowel tissue, liver, kidney, pancreas, lung, trachea, eye, cartilage tissue, and tendon tissue. In some embodiments, the biodegradable adhesive material is suitable for application to a surface that is wet, dynamically moving, or a combination of wet and dynamically moving. In some embodiments, the surface is a medical device.

In some embodiments, the hydrogel encapsulates the medical device. In an embodiment, the medical device selected from the group consisting of a defibrillator, a pacemaker, a stent, a catheter, a tissue implant, a screw, a pin, a plate, a rod, an artificial joint, a pneumatic actuator, a sensor, an elastomer-based device, and a hydrogel based device.

In some embodiments, the hydrogel is adhered to a surface in order to close a wound. In a particular embodiment, the hydrogel is adhered to a surface for a biosurgical application.

In an aspect, the invention discloses a method of delivering a therapeutically active agent to a subject, the method comprising: (a) applying a solution comprising a high density primary amine polymer and a coupling agent to a hydrogel; and (b) placing the hydrogel on a surface in the subject; wherein the hydrogel comprises a first polymer network and a second polymer network, wherein the first polymer network comprises a first polymer covalently crosslinked with a biodegradable covalent crosslinker and the second polymer network comprises a second polymer crosslinked with ionic or physical crosslinks, and wherein at least one therapeutically active agent is encapsulated in, or attached to the surface of, the hydrogel and/or high density primary amine polymer, thereby delivering a therapeutically active agent to the subject.

In another aspect, the invention discloses a biodegradable adhesive material comprising: (a) a biodegradable IPN hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks; (b) a high density primary amine polymer; and (c) a coupling agent, wherein the high density primary amine polymer and the coupling agent are applied to one side of the hydrogel.

In some embodiments, the biodegradable adhesive material is in the form of a preformed patch. In some embodiments, the biodegradable adhesive material is in the form of an injectable gel.

In some embodiments of the biodegradable adhesive material, the first polymer network is modified with two reactive moieties, wherein the reactive moieties are each independently selected from the group consisting of methacrylate, acrylate, acrylamide, methacrylamide, thiol, hydrazine, tetrazine, norbornene, transcyclooctene and cyclooctyne.

In some embodiments of the biodegradable adhesive material, the second polymer network is alginate.

In some embodiments of the biodegradable adhesive material, the first polymer network comprises polyethylene glycol (PEG) modified with norbornene and polyethylene glycol (PEG) modified with tetrazine.

In some embodiments of the biodegradable adhesive material, the two reactive moieties react in the presence of Ca²⁺ (e.g., CaSO₄). In some embodiments of the biodegradable adhesive material, the two reactive moieties react in the presence of UV light.

The present invention is illustrated by the following drawings and detailed description, which do not limit the scope of the invention described in the claims.

### BRIEF DESCRIPTION THE DRAWINGS

FIG. 1 is a plot comparing fracture energy values for tough gels with different percent weight concentration of PEGDA 250 covalent crosslinker. Data shown as mean±standard deviation. N=3/group.

FIG. 2 is a plot comparing fracture energy values for tough gels with different percent weight concentration of PEGDA 10 k covalent crosslinker. Data shown as mean±standard deviation. N=3/group. 2-way ANOVAs with post hoc t-test.

FIG. 3 is a plot comparing fracture energy values for tough gels with different percent weight concentration of PEGDA 250 and PEGDA 10 k covalent crosslinkers. Data shown as mean±standard deviation. N=3/group.

FIG. 4 is a plot comparing fracture energy values for tough gels with different percent weight concentration of GelMA covalent crosslinker. Data shown as mean±standard deviation. N=3/group. 2-way ANOVAs with post hoc t-test.

FIG. 5 is a plot comparing fracture energy values for tough gels with different percent weight concentration of AlgMA-5 Mrad covalent crosslinker. Data shown as mean±standard deviation. N=3/group.

FIG. 6 is a plot comparing fracture energy values in kJ/m² for best performing concentration of each crosslinker. Data shown as mean±standard deviation. N=3/group. 2-way ANOVAs with post hoc t-test.

FIGS. 7A, 7B, and 7C are plots comparing the mechanical properties (stress, stretch, toughness) of tough gels having 1× or 2× concentration of the hydrolyzable covalent crosslinker bis(2-methacryloyl)oxyethyl disulfide (Bis).

FIGS. 8A, 8B, and 8C are plots comparing the mechanical properties (stress, stretch, toughness) of tough gels having 1× or 2× concentration of the reduction-cleavable covalent crosslinker N,N′-Bis(acryloyl)cystamine (Cys).

FIGS. 9A, 9B, and 9C are plots comparing the mechanical properties (stress, stretch, toughness) of tough gels having 1× or 2× concentration of the enzymatically-cleavable covalent crosslinker gelatin methacrylate (GeIMA).

FIGS. 10A, 10B, and 10C are plots comparing the mechanical properties (stress, stretch, toughness) of tough gels having 1× or 2× concentration of the enzymatically-cleavable covalent crosslinker hyaluronic acid methacrylate (HAMA).

FIGS. 11A, 11B, and 11C are plots comparing the mechanical properties (stress, stretch, toughness) of tough gels having 1× or 2× concentration of the hydrolyzable covalent crosslinker oxidized alginate methacrylate (OxAlgMA).

FIGS. 12A, 12B, and 12C are plots comparing the mechanical properties (stress, stretch, toughness) of tough gels having 1× or 2× concentration of the hydrolyzable covalent crosslinker poly(ethylene glycol) diacrylate 250 (PEGDA 250).

FIGS. 13A, 13B, and 13C are plots comparing the mechanical properties (stress, stretch, toughness) of tough gels having 1× or 2× concentration of the hydrolyzable covalent crosslinker poloxamer diacrylate (Polox DA).

FIGS. 14A, 14B, and 14C are plots comparing the mechanical properties (stress, stretch, toughness) of tough gels having the biodegradable covalent crosslinkers Bis, Cys, and GelMA, HAMA, OxAlgMa, PEGDA 250, and Polox DA.

FIG. 15 is a plot comparing the mass loss percentages for tough gels having a non-biodegradable covalent crosslinker, or PEGDA 10 k and GelMA biodegradable covalent crosslinkers.

FIGS. 16A, 16B, and 16C evaluate the degradation of tough gels having different biodegradable covalent crosslinkers (GelMA, HAMA, OxAlgMa, PEGDA 250, and Polox DA) over a period of 16 weeks, through the measurement of percentage of the gel recovered, gel thickness, and gel mass. (compared to tough gels having MBAA non-degradable covalent crosslinker)

FIG. 17 is a series of high frequency hematoxylin- and eosin-stained (HE stain) images of subcutaneously implanted tough gels (with alginate or oxidized alginate) having the control non-biodegradable MBAA crosslinker at 1 week, 2 weeks, 4 weeks, 8 weeks, and 16 weeks.

FIG. 18 is a series of high frequency hematoxylin- and eosin-stained (HE stain) images of subcutaneously implanted tough gels (with alginate or oxidized alginate) having the biodegradable PEGDA 250 crosslinker at 1 week, 2 weeks, 4 weeks, 8 weeks, and 16 weeks.

FIG. 19 is a series of high frequency hematoxylin- and eosin-stained (HE stain) images of subcutaneously implanted tough gels (with alginate or oxidized alginate) having the biodegradable Polox DA crosslinker at 1 week, 2 weeks, 4 weeks, and 8 weeks.

FIG. 20 is a series of high frequency hematoxylin- and eosin-stained (HE stain) images of subcutaneously implanted tough gels (with alginate or oxidized alginate) having the biodegradable HAMA crosslinker at 4 weeks, 8 weeks, and 16 weeks.

FIG. 21 is a series of high frequency hematoxylin- and eosin-stained (HE stain) images of subcutaneously implanted tough gels (with alginate or oxidized alginate) having the biodegradable GelMA crosslinker at 4 weeks, 8 weeks, and 16 weeks.

FIG. 22 is a series of high frequency hematoxylin- and eosin-stained (HE stain) images of subcutaneously implanted tough gels (with alginate or oxidized alginate) having the biodegradable OxAlgMA crosslinker at 4 weeks, 8 weeks, and 16 weeks.

FIGS. 23A, 23B, 23C, 23D, 23E, and 23F are plots comparing effects in tough gel tensile mechanical properties after 1 minute of treatment with various chemical or enzymatic solutions.

FIGS. 24A and 24B are plots comparing the effect of various solutions on tough gel tensile mechanical properties (toughness and maximum stress).

FIGS. 25A and 25B are plots comparing the effect of alginate lyase treatment on tough gel mechanical properties (toughness and maximum stress) over a period of 100 minutes.

FIGS. 26A and 26B are high frequency ultrasound and hematoxylin- and eosin-stained (HE stain) images of skin on the back of a mouse (control); skin with tough gel adhesive, skin with with tough gel adhesive and alginate lyase treatment; skin with Dermabond adhered then peeled; and skin with cyanoacrylate adhered then peeled.

### DETAILED DESCRIPTION OF THE INVENTION

The present invention discloses biodegradable interpenetrating networks (IPN) hydrogels. The present invention is based, at least in part, on the discovery of biodegradable tough adhesive materials that are capable of adhering to biological surfaces (for example, tissue) even in wet and dynamic environments. Accordingly, the present invention provides compositions and methods of adhering a biodegradable tough adhesive material comprising a biodegradable interpenetrating networks hydrogel to a biological surface.

The biodegradable tough adhesive materials described herein offer significant advantages in medical applications, including wound dressings, biosurgical applications, drug delivery and tissue repair. For example, hydrogels that are used on wet, dynamic tissues, such as muscles or the heart, are subject to application of repeated stresses and strains. Since the biodegradable hydrogels described herein are more mechanically robust, more durable, and are characterized by a higher interfacial toughness, they are more suitable for such applications.

### I. Definitions

In order that the present invention may be more readily understood, certain terms are first defined. In addition, it should be noted that whenever a value or range of values of a parameter are recited, it is intended that values and ranges intermediate to the recited values are also part of this invention.

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to phrases such as “one embodiment” or “an embodiment” means that a particular feature, structure or characteristic described in connection with the embodiment is included in at least one embodiment of the invention. The appearances of phrases such as “in one embodiment” in various places in the specification are not necessarily all referring to the same embodiment.

The articles “a” and “an” are used herein to refer to one or to more than one (i.e., to at least one) of the grammatical object of the article. By way of example, “an element” means one element or more than one element.

As used herein the term “comprising” or “comprises” is used in reference to compositions, methods, and respective component(s) thereof, that are essential to the invention, yet open to the inclusion of unspecified elements, whether essential or not.

The term “consisting of” refers to compositions, methods, and respective components thereof as described herein, which are exclusive of any element not recited in that description of the embodiment.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein. Preferably, the subject is a mammal. The mammal can be a human, non-human primate, mouse, rat, dog, cat, horse, or cow. Mammals other than humans can be advantageously used as subjects that represent animal models of tissue or organ injuries, or other related pathologies. A subject can be male or female. The subject can be an adult, an adolescent or a child. A subject can be one who has been previously diagnosed with or identified as suffering from or having a risk for developing a tissue injury, disease or condition associated with tissue injury, or requires a device to be attached within or onto the body of the subject.

### II. Compositions of the Invention

### A. Biodegradable Interpenetrating Networks Hydrogels

The present invention provides a composition comprising a biodegradable IPN hydrogel, comprising a first polymer network and a second polymer network, wherein the first polymer network comprises a first polymer covalently crosslinked with a biodegradable covalent crosslinker and the second polymer network comprises a second polymer crosslinked with ionic or physical crosslinks. Surprisingly, the IPN hydrogels of the present invention show high mechanical strength and tunable biodegradability.

In one embodiment, the present invention provides a composition comprising a biodegradable IPN hydrogel comprising a first polymer network and a second polymer network, wherein the first polymer network comprises polyacrylamide and a biodegradable covalent crosslinker PEGDA, and the second polymer network comprises an ionically cross-linked alginate polymer.

A biodegradable covalent crosslinker, as used herein, is a biodegradable compound or polymer having one or more acrylate moieties. The acrylate moiety as used herein is selected from the group consisting of alkylated acrylate, e.g., methyl acrylate (methacrylate), dimethyl acrylate, ethyl acrylate etc., monoacrylate (acrylate) and diacrylate. In some embodiments, the biodegradable covalent crosslinker comprising a biodegradable acrylated polymer is selected from the group consisting of an acrylated polysaccharide, an acrylated protein, an acrylated polyester, an acrylated polyol (polyalcohol) and an acrylated polyether, or a combination thereof, wherein the polysaccharide, the protein, the polyol and the polyether may be optionally oxidized. Exemplary biodegradable acrylated polymers include a poly(ethylene glycol) acrylate, a gelatin acrylate, a hyaluronic acid acrylate, a polycaprolactone (PCL) acrylate, a poly(lactide)-poly(ethylene glycol)-poly(lactide) (PLA-PEG-PLA) acrylate and an alginate acrylate. In some embodiments, the biodegradable covalent crosslinker is selected from the group consisting of a polycaprolactone dimethacrylate, a poly(ethylene glycol) diacrylate (PEGDA), a poly(lactide)-poly(ethylene glycol)-poly(lactide) diacrylate (Acrylate-PLA-PEG-PLA-Acrylate), a poly(d,l-lactide)-poly(ethylene glycol)-poly(d,l-lactide) dimethacrylate (MA-PDLLA-PEG-PDLLA-MA), a gelatin methacrylate (GeIMA), a methacrylated alginate (AlgMA), an oxidized, methacrylated alginate (OxAlgMA) and an AlgMA-5 Mrad with a molecular weight from about 100 Da to about 40,000 Da. In some embodiments, the biodegradable covalent crosslinker comprises a biodegradable acrylated compound, for example, diurethane dimethacrylate, bis(2-(methacryloyloxy)ethyl) phosphate, glycerol dimethacrylate and ethylene glycol diacrylate.

In some embodiments, the biodegradable covalent crosslinker is selected from the group consisting of a poly(ethylene glycol) acrylate, a gelatin acrylate, a hyaluronic acid acrylate, an alginate acrylate a poloxamer acrylate, and a disulfide-based crosslinker. In some embodiments, the biodegradable covalent crosslinker is selected from the group consisting of a poly(ethylene glycol) acrylate, a gelatin acrylate, a hyaluronic acid acrylate and an alginate acrylate. In some embodiments, the biodegradable covalent crosslinker is selected from the group consisting of a poly(ethylene glycol) diacrylate (PEGDA), gelatin methacrylate (GeIMA), alginate methacrylate (AlgMA), hyaluronic acid methacrylate (HAMA), a poloxamer acrylate, a disulfide-based acrylate, and N,N′-bis(acryloyl)cystamine (Cys). In some embodiments, the biodegradable covalent crosslinker is selected from the group consisting of poly(ethylene glycol) diacrylate 250 (PEGDA 250), gelatin methacrylate (GeIMA), hyaluronic acid methacrylate (HAMA), oxidized alginate methacrylate (OxAlgMA), poloxamer diacrylate (Polox DA), bis(2-methacryloyl)oxyethyl disulfide (Bis), and N,N-bis(acryloyl)cystamine (Cys). In some embodiments, the biodegradable covalent crosslinker is selected from the group consisting of a poly(ethylene glycol) diacrylate (PEGDA), a gelatin methacrylate (GelMA), and a methacrylated alginate (AlgMA).

As used herein, a poloxamer is a block polymer of hydrophilic poly(ethylene oxide) (PEO) and hydrophobic poly(propylene oxide) (PPO) blocks arranged in a tri-block structure as PEO-PPO-PEO. A poloxamer acrylate is a poloxamer functionalized with one or more acrylate moieties.

In an embodiment, the concentration of the poly(ethylene glycol) diacrylate (PEGDA), such as PEGDA 250, in the hydrogel is about 0.001 wt. % to 0.05 wt. % based on the weight of the first polymer, e.g., about 0.002 wt. % to 0.05 wt. %, about 0.005 wt. % to 0.05 wt. %, 0.001 wt. % to 0.05 wt. %, 0.005 wt. % to 0.02 wt. %, 0.005 wt. % to 0.01 wt. % based on the weight of the first polymer. In another embodiment, the concentration of PEGDA 250 in the hydrogel is about 0.0015 wt. % to 0.06 wt. % based on the weight of the polyacrylamide. In a specific embodiment, the concentration of the poly(ethylene glycol) diacrylate (PEGDA), such as PEGDA 250, in the hydrogel is about 0.01 wt. % based on the weight of the first polymer, such as polyacrylamide.

In another embodiment, the concentration of PEGDA 10 k in the hydrogel is about 0.003 wt. % to 0.06 wt. % based on the weight of the polyacrylamide.

In one embodiment, the concentration of the poloxamer acrylate, such as poloxamer diacrylate (Polox DA), in the hydrogel is about 0.001 wt. % to 0.05 wt. % based on the weight of the first polymer, e.g., about 0.01 wt. % to 0.05 wt. %, 0.01 wt. % to 0.025 wt. %, 0.01 wt. % to 0.02 wt. %, 0.001 wt. % to 0.02 wt. %, 0.001 wt. % to 0.01 wt. % based on the weight of the first polymer. In a specific embodiment, the concentration of the poloxamer acrylate, such as poloxamer diacrylate (Polox DA), in the hydrogel is about 0.02 wt. % based on the weight of the first polymer, such as polyacrylamide.

In yet another embodiment, the concentration of gelatin methacrylate (GeIMA) in the hydrogel is about 0.012 wt. % to 0.2 wt. % based on the weight of the polyacrylamide. In one embodiment, the concentration of the gelatin methacrylate (GeIMA) in the hydrogel is about 0.001 wt. % to 0.05 wt. % based on the weight of the first polymer, e.g., about 0.003 wt. % to 0.01 wt. %, 0.002 wt. % to 0.05 wt. %, 0.002 wt. % to 0.02 wt. %, 0.002 wt. % to 0.01 wt. %, 0.005 wt. % to 0.01 wt. %, 0.0025 wt. % to 0.005 wt. % based on the weight of the first polymer. In a specific embodiment, the concentration of the gelatin methacrylate (GelMA) in the hydrogel is about 0.005 wt. % based on the weight of the first polymer, such as polyacrylamide.

In an embodiment, the concentration of AlgMA-5 Mrad in the hydrogel is about 0.012 wt. % to 0.2 wt. % based on the weight of the polyacrylamide.

In one embodiment, the concentration of the oxidized alginate methacrylate (OxAlgMA) in the hydrogel is about 0.001 wt. % to 0.05 wt. % based on the weight of the first polymer, e.g., about 0.002 wt. % to 0.05 wt. %, about 0.005 wt. % to 0.05 wt. %, 0.001 wt. % to 0.05 wt. %, 0.005 wt. % to 0.02 wt. %, 0.005 wt. % to 0.01 wt. % based on the weight of the first polymer. In a specific embodiment, the concentration of the oxidized alginate methacrylate (OxAlgMA) in the hydrogel is about 0.01 wt. % based on the weight of the first polymer, such as polyacrylamide.

In one embodiment, the concentration of the hyaluronic acid methacrylate (HAMA) in the hydrogel is about 0.001 wt. % to 0.05 wt. % based on the weight of the first polymer, e.g., about 0.002 wt. % to 0.05 wt. %, about 0.005 wt. % to 0.05 wt. %, 0.001 wt. % to 0.05 wt. %, 0.005 wt. % to 0.02 wt. %, 0.005 wt. % to 0.01 wt. % based on the weight of the first polymer. In a specific embodiment, the concentration of thehyaluronic acid methacrylate (HAMA) in the hydrogel is about 0.01 wt. % based on the weight of the first polymer, such as polyacrylamide.

In one embodiment, the concentration of the disulfide-based acrylate, such as bis(2-methacryloyl)oxyethyl disulfide, in the hydrogel is about 0.005 wt. % to 0.03 wt. % based on the weight of the first polymer, e.g., about 0.005 wt. % to 0.025 wt. %, 0.01 wt. % to 0.025 wt. %, 0.01 wt. % to 0.03 wt. %, 0.01 wt. % to 0.02 wt. %, 0.005 wt. % to 0.02 wt. % based on the weight of the first polymer. In a specific embodiment, the concentration of the disulfide-based acrylate, such as bis(2-methacryloyl)oxyethyl disulfide, in the hydrogel is about 0.02 wt. % based on the weight of the first polymer, such as polyacrylamide.

In one embodiment, the concentration of N,N′-bis(acryloyl)cystamine (Cys) in the hydrogel is about 0.0005 wt. % to 0.01 wt. % based on the weight of the first polymer, e.g., about 0.0005 wt. % to 0.002 wt. %, 0.0005 wt. % to 0.001 wt. %, 0.001 wt. % to 0.002 wt. %, 0.001 wt. % to 0.002 wt. % based on the weight of the first polymer. In a specific embodiment, the concentration of N,N′-bis(acryloyl)cystamine (Cys) in the hydrogel is about 0.001 wt. % based on the weight of the first polymer, such as polyacrylamide.

The term “biodegradable” as used herein, refers to the breakdown of a material safely and relatively quickly, by biological means, into raw materials of nature which disappear into the environment. Biodegradable adhesive materials (further described in Section B. below) or hydrogels disclosed herein degrade within about 12 hours, 24 hours, 2 days, 3 days, 4 days, 5 days, 6 days, 1 week, 2 weeks, 3 weeks, 4 weeks, 5 weeks, 6 weeks, 8 weeks, 10 weeks, 12 weeks, 15 weeks, 16 weeks, 20 weeks, 24 weeks, 1 month, 2 months or 3 months (e.g., based on the simulated (outside the body) hydrolytic or enzymatic solution of varying pH or enzyme). For example, the biodegradable hydrogels and adhesive materials can degrade within 1-6 days, 1-4 weeks, or 1-4 months. In some embodiments, a “biodegradable covalent crosslinker”, as used herein, refers to covalent crosslinkers that are hydrolyzable. Examples of hydrolyzable covalent crosslinkers include poly(ethylene glycol) acrylates, poloxamer acrylates, disulfide-based acrylates, alginate acrylates, oxidized alginate acrylates. In some embodiments, a “biodegradable covalent crosslinker”, as used herein, refers to covalent crosslinkers that are enzymatically cleavable. Examples of reduction-cleavable covalent crosslinkers include N,N′-bis(acryloyl)cystine and N,N′-bis(acryloyl)cystamine (Cys). In some embodiments, a “biodegradable covalent crosslinker”, as used herein, refers to covalent crosslinkers that are enzymatically cleavable. Examples of enzymatically cleavable crosslinkers include gelatin acrylates and hyaluronic acid acrylates.

Traditionally, polyacrylamide used in the first polymer network is crosslinked with a N,N-methylenebisacrylamide (MBAA) covalent crosslinker to provide high mechanical strength or toughness to the hydrogel. Surprisingly, the IPN hydrogels of the present invention comprising polyacrylamide and the biodegradable covalent crosslinkers, for example PEGDA, show very high mechanical strength or toughness and tunable biodegradability.

As used herein, an interpenetrating network (IPN) is a polymer network comprising two or more networks (e.g., a first polymer network and a second polymer network) which are at least partially interlaced on a molecular scale but not covalently bonded to each other and cannot be separated unless chemical bonds are broken. Alternatively, the first polymer network and the second polymer network are covalently coupled. This mixing leads to enhanced mechanical properties of the IPN hydrogels. The high fracture toughness of these biodegradable hydrogels is because of their ability to dissipate energy. Alginate-polyacrylamide hydrogels, as an example, possess ionic cross-links formed via electrostatic interactions between alginate and calcium ions that can break and dissipate energy under deformation. IPNs are described in International Patent Application No. WO 2013/103956 A1, which is incorporated herein by reference in its entirety.

In particular, the first polymer network comprises covalent crosslinks and includes a polymer selected from the group consisting of polyacrylamide, poly(vinyl alcohol), poly(ethylene oxide) and its copolymers, polyethylene glycol (PEG), and polyphosphazene. Also, any polymer that is methacrylated (e.g., methacrylated PEG) could be used in a similar manner. In a particular embodiment, the polymer is selected from the group consisting of polyacrylamide (PAAM), poly(hydroxyethylmethacrylate) (PHEMA), poly(vinyl alcohol) (PVA), polyethylene glycol (PEG), polyphosphazene, collagen, gelatin, poly(acrylate), poly(methacrylate), poly(methacrylamide), poly(acrylic acid), poly(N-isopropylacrylamide) (PNIPAM), poly(N,N-dimentylacrylamide), poly(allylamine) and copolymers thereof. In a particular embodiment, the first polymer is polyethylene glycol (PEG). In some embodiments, the first polymer is polyacrylamide (PAAM).

The second polymer network includes ionic crosslinks and is a polymer selected from the group consisting of alginate (alginic acid or align), pectate (pectinic acid or polygalacturonic acid), carboxymethyl cellulose (CMC or cellulose gum), hyaluronate (hyaluronic acid or hyaluronan), chitosan, ι-carrageenan, ι-carrageenan and λ-carrageenan, wherein the wherein the alginate, carboxymethyl cellulose, hyaluronate, chitosan, κ-carrageenan, ι-carrageenan and λ-carrageenan are each optionally oxidized, wherein the alginate, hyaluronate, chitosan, κ-carrageenan, ι-carrageenan and λ-carrageenan optionally include one or more groups selected from the group consisting of methacrylate, acrylate, acrylamide, methacrylamide, thiol, hydrazine, tetrazine, norbornene, transcyclooctene and cyclooctyne. Crosslinkers that promote ionic crosslinks include CaCl₂, CaSO₄, CaCO₃, hyaluronic acid, and polylysine.

In a particular embodiment, the second polymer network is alginate, which is comprised of (1-4)-linked b-D-mannuronic acid (M) and a-L-guluronic acid (G) monomers that vary in amount and sequential distribution along the polymer chain. Alginate is also considered a block copolymer, composed of sequential M units (M blocks), regions of sequential G units (G blocks), and regions of alternating M and G units (M-G blocks) that provide the molecule with its unique properties. Alginates have the ability to bind divalent cations such as Ca⁺² between the G blocks of adjacent alginate chains, creating ionic interchain bridges between flexible regions of M blocks. In some embodiments, the alginate is a mixture of a high molecular weight alginate and a low molecular weight alginate. For example, the ratio of the high molecular weight alginate to the low molecular weight alginate is about 0% and 100%; about 10% and 90%; about 20% and 80%; about 30% and 70%; about 40% and 60%; about 50% and 50%; about 60% and 40%; about 70% and 30%; about 80% and 20%; about 90% and 10%; about 100% and 0%. The high molecular weight alginate has a molecular weight from about 100,000 Da to about 300,000 Da, from about 150,000 Da to about 250,000 Da, or is about 200,000 Da. The low molecular weight alginate has a molecular weight from about 1,000 Da to about 100,000 Da, from about 5,000 Da to about 50,000 Da, from about 10,000 Da to about 30,000 Da, or is about 20,000 Da.

The hydrogels of the invention are highly absorbent and comprise about 30% to about 98% water (e.g., about 40%, about, about 50%, about 60%, about 70%, about 80%, about 90%, about 95%, about 98%, about 40 to about 98%, about 50 to about 98%, about 60 to about 98%, about 70 to about 98%, about 80 to about 98%, about 90 to about 98%, or about 95 to about 98% water) and possess a degree of flexibility similar to natural tissue, due to their significant water content. In particular, the hydrogels of the present invention can be stretched up to 20 times their initial length, e.g., the hydrogels of present invention can be stretched from 2 to 20 times their initial length, 5 to 20 times their initial length, 10 to 20 times their initial length, from 15 to 20 times their initial length, from 2 to 10 times their initial length, from 10 to 15 times their initial length, and from 5 to 15 times their initial length without cracking or tearing.

Hydrogels with high fracture energies (toughness) are more mechanically robust than hydrogels with low fracture energies (toughness). The biodegradable IPN hydrogels of the invention comprise a fracture toughness value of between 2.5 kJ/m² and 20 kJ/m², e.g., between 10 kJ/m² and 20 kJ/m², between 12 kJ/m² and 20 kJ/m², between 13 kJ/m² and 20 kJ/m² or between 15 kJ/m² and 20 kJ/m². The interpenetrating polymer network comprises a fracture toughness value of at least 5 kJ/m², at least 10 kJ/m², at least 10 kJ/m², or at least 20 kJ/m². In preferred embodiments, the interpenetrating polymer network comprises a fracture toughness value of at least 10 kJ/m², at least 11 kJ/m², at least 12 kJ/m², at least 13 kJ/m², at least 14 kJ/m², at least 15 kJ/m², at least 16 kJ/m², at least 17 kJ/m², at least 18 kJ/m², at least 19 kJ/m² or at least 20 kJ/m². Hydrogels with high fracture toughness are able to withstand large deformations prior to rupture. This may be important to dissipate mechanical energy and withstand cyclic fatigue loading. The adhesion energy for these tough gels with different crosslinkers may be measured with peeling tests, where the tough adhesive is adhered to the tissue surface with one end open.

In order to increase the fracture toughness of the interpenetrating polymer network, the hydrogel may be cured at a temperature of between 20° C. and 100° C., e.g., between 40° C. and 90° C., between 60° C. and 80° C., or about 70° C. For example, the hydrogel is cured at a temperature of between 20° C. and 36° C., e.g., 21° C., 22° C., 23° C., 24° C., 25° C., 26° C., 27° C., 28° C., 29° C., 30° C., 31° C., 32° C., 33° C., 34° C., or 35° C. In other examples, the curing is carried out at about 50° C. This thermal treatment is performed before free radical polymerization. In some examples, the curing is carried out at freezing temperatures, for example, from about about 0° C. to about −30° C. to induce porosity. The mixture of alginate and acrylamide is cured at a selected temperature for at least 10 min., 20 min., 30 min., 45 min., 60 min, 90 min., or 120 min.

The polymer ratio between the first polymer, e.g., the polyacrylamide polymer, and the second polymer, e.g., the alginate polymer, is between about 66.67 wt. % and 94.12 wt. %, about 88.89 wt. % or about 85.71 wt. %.

In some cases, the ratio between CaSO₄ and alginate is between about 3.32 wt. % and 53.15 wt. %, e.g., about 13.28 wt. %.

The biodegradable IPN hydrogel comprises a biodegradable covalent crosslinker/first polymer, e.g., acrylamide, with a weight ratio between about 0.0015 wt. % and 0.2 wt. %, between about 0.006 wt. % and 0.06 wt. %, between about 0.0015 wt. % and 0.06 wt. %, between about 0.012 wt. % and 0.2 wt. % or about 0.003 wt. %.

The biodegradable IPN hydrogel can undergo hydrolytic or enzymatic degradation. The biodegradable IPN hydrogel undergoes hydrolytic degradation after incubation for at least 12 hours, 24 hours, 48 hours, 3 days, 4 days, 5 days, 6 days, or 7 days in an accelerated hydrolytic solution. In some cases, the gels can pass through a 30G needle within 24 h of incubation in the solution.

### B. Biodegradable Tough Adhesive Material

The present invention also provides a composition comprising a biodegradable tough adhesive material, comprising: (a) a biodegradable IPN hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks; (b) an adhesive bridging polymer; and (c) a coupling agent.

The biodegradable tough adhesive material provides an adhesive surface to the biodegradable IPN hydrogel. The adhesive surface comprises interpenetrating positively charged polymers, and the hydrogel provides a bulk matrix (also referred to as a dissipative matrix) that can dissipate energy effectively under deformation. The adhesive surface can form electrostatic interactions, covalent bonds, and physical interpenetration with an adherent surface of a substrate (e.g., a tissue, a cell, or a device), while the bulk matrix dissipates energy through hysteresis under deformation. For example, for substrates that bear functional groups like amines and carboxylic acids, adhesion can be formed via electrostatic interactions and covalent bonds between the biodegradable tough adhesive (TA) and the substrate. For substrates that are hydrophilic and permeable to macromolecules, the high density primary amine polymers (also referred to herein as “bridging plymers”) can interpenetrate into the substrate forming physical entanglements, and also form covalent bonds with the tough gel adhesive matrix. When an interface is stressed, the matrix dissipates energy by breaking ionic cross-links. The combination is designated to achieve high adhesion energy and bulk toughness simultaneously. The tough adhesive compositions are described in detail in the International Patent Application No. WO 2017/165490 A1, which is incorporated herein by reference in its entirety.

In some embodiments, the hydrogel is fabricated in the form of a patch. The patch can either be preformed and ready to be applied to a surface or the patch can be cut to the desired size and shape prior to application.

Alternatively, in some embodiment, the biodegradable adhesive material of the present invention may be delivered by injection. Water soluble sodium alginate readily binds calcium, forming an insoluble calcium alginate hydrocolloid (Sutherland, 1991, Biomaterials, Palgrave Macmillan UK:307-331). These gentle gelling conditions have made alginate a popular material as an injectable cell delivery vehicle (Atala et al., 1994, J. Urol. 152(2 Pt 2):641-3). Accordingly, in some embodiments, the biodegradable adhesive material is suitable for injection into a subject. Injectable adhesives may include a polymer that includes at least two reactive moieties that react and form the first polymer network upon injection. The two reactive moieties may be present on each polymer or the polymer is made of two populations of polymers, each one with a different reactive moiety. Exemplary reactive moieties include methacrylate, acrylate, acrylamide, methacrylamide, thiol, hydrazine, tetrazine, norbornene, transcyclooctene and cyclooctyne. In a particular embodiment, the two reactive moieties react in the presence of UV light. In a particular embodiment, the two reactive moieties react in the presence of Ca²⁺ (e.g., CaSO₄).

The biodegradable adhesive material includes a high density primary amine polymer (also referred to herein as a “bridging polymer”). The high density primary amine polymer forms covalent bonds with both the hydrogel and the surface, bridging the two. The high density primary amine polymer bears positively charged primary amine groups under physiological conditions. In some embodiments, the high density primary amine polymer can be absorbed to a surface (e.g., a tissue, a cell, or a device) via electrostatic interactions, and provide primary amine groups to bind covalently with both carboxylic acid groups in the hydrogel and on the surface. If the surface is permeable, the high density primary amine polymer can also penetrate into the surface, forming physical entanglements, and then chemically anchor the hydrogel.

As used herein, the high density primary amine polymer includes at least one primary amine per monomer unit. In some embodiments, the high density primary amine polymer is selected from the group consisting of chitosan, gelatin, collagen, polyallylamine, polylysine, and polyethylenimine. In particular, chitosan is represented by the following structural formula:

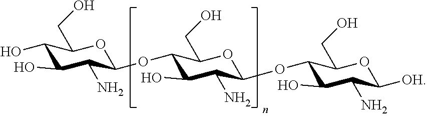

The biodegradable adhesive material also includes a coupling agent. As used herein, the coupling agent activates one or more of the primary amines present in the high density primary amine polymer. Once activated with the coupling agent, the primary amine forms an amide bond with the hydrogel and the target surface (e.g., a tissue, an organ, or a medical device). In some embodiments, the coupling agent includes a first carboxyl activating agent, wherein the first carboxyl activating agent is a carbodiimide. Exemplary carbodiimides are selected from the group consisting of 1-ethyl-3-(3-dimethylaminopropyl)carbodiimide (EDC, EDAC or EDCI), dicyclohexylcarbodiimide (DCC) and diisopropylcarbodiimide (DIC). In some embodiments, the first carboxyl activating agent is EDC.

In some embodiments, the coupling agent further includes a second carboxyl activating agent. Exemplary second carboxyl activating agents include, but are not limited to, N-hydroxysuccinimide (NHS), N-hydroxysulfosuccinimide (sulfo-NHS), hydroxybenzotriazole (HOBt), dimethylaminopyridine (DMAP), Hydroxy-3,4-dihydro-4-oxo-1,2,3-benzotriazine (HOOBt/HODhbt), 1-Hydroxy-7-aza-1H-benzotriazole (HOAt), Ethyl 2-cyano-2-(hydroximino)acetate, Benzotriazol-1-yloxy-tris(dimethylamino)-phosphonium hexafluorophosphate (BOP), Benzotriazol-1-yloxy-tripyrrolidino-phosphonium hexafluorophosphate, 7-Aza-benzotriazol-1-yloxy-tripyrrolidinophosphonium hexafluorophosphate), Ethyl cyano(hydroxyimino)acetato-02)-tri-(1-pyrrolidinyl)-phosphonium hexafluorophosphate, 3-(Diethoxy-phosphoryloxy)-1,2,3-benzo[d] triazin-4(3H)-one, 2-(1H-Benzotriazol-1-yl)-N,N,N′,N′-tetramethylaminium tetrafluoroborate/hexafluorophosphate, 2-(6-Chloro-1H-benzotriazol-1-yl)-N,N,N′,N′-tetramethylaminium hexafluorophosphate), N-[(5-Chloro-1H-benzotriazol-1-yl)-dimethylamino-morpholino]-uronium hexafluorophosphate N-oxide, 2-(7-Aza-1H-benzotriazol-1-yl)-N,N,N′,N′-tetramethylaminium hexafluorophosphate, 1-[1-(Cyano-2-ethoxy-2-oxoethylideneaminooxy)-dimethylamino-morpholino]-uronium hexafluorophosphate, 2-(1-Oxy-pyridin-2-yl)-1,1,3,3-tetramethylisothiouronium tetrafluoroborate, Tetramethylfluoroformamidinium hexafluorophosphate, N-Ethoxycarbonyl-2-ethoxy-1,2-dihydroquinoline, 2-Propanephosphonic acid anhydride, 4-(4,6-Dimethoxy-1,3,5-triazin-2-yl)-4-methylmorpholinium salts, (bis-Trichloromethylcarbonate, 1,1′-Carbonyldiimidazole. In some embodiments, the first carboxyl activating agent is NHS.

In some embodiments, the high density primary amine polymer and the coupling agent are packaged separately.

In some embodiments, the high density primary amine polymer is in a solution and the coupling agent is in solid form. In a particular, the coupling agent is added to the high density primary amine polymer solution. In some embodiments, the high density primary amine polymer is in a solution, the coupling agent is added to the high density primary amine polymer solution, and the solution is applied to the hydrogel.

In some embodiments, the concentration of the high density primary amine polymer in the solution is about 0.1% to about 50%, for example, from about 0.2% to about 40%, about 0.5% to about 30%, about 1.0% to about 20%, about 1% to about 10%, about 0.2% to about 10%, about 10% to about 20%, about 20% to about 30%, or about 40% to about 50%. In some embodiments, the coupling agent includes at least a first carboxyl activating agent and optionally a second carboxyl activating agent, and wherein the concentration of the first carboxyl activating agent in the solution is about 3 mg/ml to about 50 mg/ml, for example from about 5 mg/ml to about 40 mg/ml, about 7 mg/ml to about 30 mg/ml, about 9 mg/ml to about 20 mg/ml, about 3 mg/ml to about 45 mg/ml, 3 mg/ml to about 40 mg/ml, 3 mg/ml to about 35 mg/ml, about 3 mg/ml to about 30 mg/ml, 3 mg/ml to about 25 mg/ml, about 3 mg/ml to about 20 mg/ml, 3 mg/ml to about 15 mg/ml, about 3 mg/ml to about 10 mg/ml, about 5 mg/ml to about 50 mg/ml, about 10 mg/ml to about 50 mg/ml, about 15 mg/ml to about 50 mg/ml, about 20 mg/ml to about 50 mg/ml, about 25 mg/ml to about 50 mg/ml, about 30 mg/ml to about 50 mg/ml, about 35 mg/ml to about 50 mg/ml, about 40 mg/ml to about 50 mg/ml, or about 3 mg/ml to about 45 mg/ml.

In some embodiments, the adhesive material includes a first therapeutically active agent. The first therapeutically active agent may be encapsulated in or attached to the surface of the hydrogel. Alternatively, the first therapeutically active agent is encapsulated in or attached to the surface of the high density primary amine polymer. In certain embodiments, the adhesive material further comprises a second therapeutically active agent. The second therapeutically active agent is encapsulated in or attached to the surface of the hydrogel. Alternatively, the second therapeutically active agent is encapsulated in or attached to the surface of the high density primary amine polymer. The first and second therapeutically active agents are independently selected from the group consisting of a small molecule, a biologic, a nanoparticle, and a cell. The biologic is selected from the group consisting of a growth factor, an antibody, a vaccine, a cytokine, a chemokine, a hormone, a protein, and a nucleic acid. The amount of therapeutically active agents included in a composition of the invention depends on various factors including, for example, the specific agent; function which it should carry out; required period of time for release of the agent; quantity to be administered. Generally, dosage of a therapeutically active agents, i.e., amount of therapeutically active agents in the system, is selected from the range of about 0.001% (w/w) to about 10% (w/w); about 1% (w/w) to about 5% (w/w); or about 0.1% (w/w) to about 1% (w/w).

The present invention also provides a biodegradable adhesive material to encapsulate a device, or to coat a surface of a device. In particular, the hydrogel and the high density primary amine polymer and coupling agent are applied to the exterior surface of the hydrogel, and then the hydrogel is applied to the surface of the device. The coupling agent and the high density primary amine polymer adhere the hydrogel to the surface of the device. Depending upon to desired outcome, the device can be completely encapsulated by the hydrogel or partially encapsulated, leaving some surface of the device exposed. Specifically, a “partially encapsulated” device refers to coating the device either on one surface of the device (e.g., the back, front or sides of the device) or on one portion of the device (e.g., the bottom half or the top half). In a particular embodiment, the high density primary amine polymer and coupling agent may be applied to multiple sites of the hydrogel so that the hydrogel can adhere to both the device and also another surface (e.g., a tissue or organ). Exemplary medical devices include, but are not limited to a defibrillator, a pacemaker, a stent, a catheter, a tissue implant, a screw, a pin, a plate, a rod, an artificial joint, a elastomer-based (e.g., PDMS, PTU) device, a hydrogel-based device (e.g., scaffolds for drug or cell delivery or sensors), and sensors for measuring, for example, temperature, pH, and local tissue strains.

A surface can have functional groups (e.g., amine or carboxylic acid groups) or can be chemically inert. The biodegradable adhesive material of the invention can form electrostatic interactions, covalent bonds, and physical interpenetration with adherent surfaces. For substrates that bear functional groups like amines and carboxylic acids, adhesion can be formed via electrostatic interactions and covalent bonds between the tough gel adhesive and the substrate. For substrates that are hydrophilic and permeable to macromolecules, the high density primary amine polymers can interpenetrate into the substrate forming physical entanglements, and also form covalent bonds with the tough gel adhesive matrix.

The interfacial adhesion between the hydrogel and the surface (e.g., tissue or device) impacts the mechanical strength and reliability of the hydrogel, which corresponds to the performance of the hydrogel as an adhesive. The nature of this interaction can be measured as the interfacial fracture toughness. Methods to measure the interfacial fracture toughness are known to those of skill in the art.

In some embodiments, the biodegradable adhesive material is transparent, allowing for ease of monitoring the surface below or the device encapsulated within.

In some embodiments, the biodegradable adhesive material is suitable for application to a surface that is wet, dynamic, or a combination of wet and dynamic. The biodegradable tough adhesive material may serve as a tool for many medical treatments requiring invasive procedures that range between suture replacements to waterproof sealants for hollow organ anastomosis, and hemostatic wound healing.

### III. Methods Of the Invention

The present invention provides a method of making a biodegradable IPN hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks. The method includes mixing a first polymer, e.g., an alginate, and a second polymer, e.g., an acrylamide polymer; and contacting the mixture with a biodegradable covalent crosslinker and an ionic crosslinker thereby making an IPN hydrogel.

The present invention also provides a method of adhering a biodegradable IPN hydrogel to a surface. The method includes the steps of a) applying a solution comprising a high density primary amine polymer and a coupling agent to the biodegradable IPN hydrogel; and b) placing the biodegradable IPN hydrogel on the surface; wherein the hydrogel comprises a first polymer network and a second polymer network, wherein the first polymer network comprises a first polymer covalently crosslinked with a biodegradable covalent crosslinker and the second polymer network comprises a second polymer crosslinked with ionic or physical crosslinks.

In certain embodiments, the surface is a tissue. The material can be applied to any tissue, including, but not limited to, heart tissue, skin tissue, blood vessel tissue, bowel tissue, liver tissue, kidney tissue, pancreatic tissue, lung tissue, trachea tissue, eye tissue, cartilage tissue, tendon tissue.

The coupling agent in solid form may be added to an aqueous solution of the high density primary amine polymer and mixed for a specified period of time, e.g., 10 seconds, 30 seconds, 60 seconds, 2 minutes, 5 minutes, or 10 minutes. This solution is then applied to the hydrogel. The treated side of the hydrogel is then placed upon the surface, e.g., tissue, causing the hydrogel to adhere due to the formation of covalent bonds between the hydrogel, the high density amine polymer and the surface.

Alternatively, the surface is a medical device. The material can be applied to any device, including, but not limited to, the group consisting of a defibrillator, a pacemaker, a stent, a catheter, a tissue implant, a screw, a pin, a plate, a rod, an artificial joint, a elastomer-based (e.g., PDMS, PTU) device, a hydrogel-based device (e.g., scaffolds for drug or cell delivery or sensors), and sensors for measuring, for example, temperature, pH, and local tissue strains.

As used herein, the term “contacting” (e.g., contacting a surface) is intended to include any form of interaction of a hydrogel and a surface (e.g., tissue or device). Contacting a surface with a composition may be performed either in vivo or ex vivo. In certain embodiments, the surface is contacted with the biodegradable adhesive material ex vivo and subsequently transferred into a subject. Alternatively, the surface is contacted with the biodegradable adhesive material in vivo. Contacting the surface with the biodegradable adhesive material in vivo may be done, for example, by injecting the biodegradable adhesive material into the surface, or by injecting the biodegradable adhesive material into or around the surface.

The present invention also includes methods to encapsulate a medical device, or to coat a surface of a device. In particular, the biodegradable IPN hydrogel and the high density primary amine polymer and coupling agent are applied to the exterior surface of the hydrogel, and then the hydrogel is applied to the surface of the device. The coupling agent and the high density primary amine polymer adhere the hydrogel to the surface of the device. Depending upon to desired outcome, the device can be completely encapsulated by the hydrogel or partially encapsulated, leaving some surface of the device exposed. Specifically, a “partially encapsulated” device refers to coating the device either on one surface of the device (e.g., the back, front or sides of the device) or on one portion of the device (e.g., the bottom half or the top half). In a particular embodiment, the high density primary amine polymer and coupling agent may be applied to multiple sites of the hydrogel so that the hydrogel can adhere to both the device and also another surface (e.g., a tissue).

The present invention also includes a method to close a wound or injury and promote wound healing. In particular, the biodegradable IPN hydrogel and the high density primary amine polymer and coupling agent are applied to the exterior surface of the hydrogel, and then the hydrogel is applied to the location of the wound or injury. In a particular embodiment, the biodegradable IPN hydrogel is applied to the heart in order to repair a heart defect.

The present invention also includes methods of delivering a therapeutically active agent to a subject. The methods include a) applying a solution comprising a high density primary amine polymer and a coupling agent to a biodegradable IPN hydrogel; and b) placing the biodegradable IPN hydrogel on the surface; wherein the biodegradable IPN hydrogel comprises a first polymer network and a second polymer network, wherein the first polymer network comprises a first polymer covalently crosslinked with a biodegradable covalent crosslinker and the second polymer network comprises a second polymer crosslinked with ionic or physical crosslinks, and wherein at least one therapeutically active agent is encapsulated in, or attached to the surface of, the hydrogel and/or high density primary amine polymer, thereby delivering a therapeutically active agent to the subject.

The methods of the present invention include contacting a surface, e.g., a tissue or a device, with a biodegradable adhesive material of the invention. The surface can be contacted with the composition by any known routes in the art. As used herein, the term “delivery” refers to the placement of a composition of the invention into a subject by a method or route which results in at least partial localization of the composition at a desired site such that a desired effect is produced.

Exemplary modes of delivery include, but are not limited to, injection, insertion, implantation, or delivery within a scaffold that encapsulates the composition of the invention at the target surface, e.g., a tissue or organ. When the compositions of the invention are dissolved in a solution, they can be injected into the surface by a syringe.

The methods of the present invention are suitable for medical purposes, e.g., wound closure, biosurgery applications, delivery of a therapeutic agent, or attachment of a medical device, in a subject, wherein the subject is a mammal. In some embodiments, a mammal is a primate, e.g., a human or an animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, a subject is selected from the group consisting of a human, a dog, a pig, a cow, a rabbit, a horse, a cat, a mouse and a rat. In preferred embodiments, the subject is a human.

As used herein the term “biosurgery” refers to the use of natural or manmade materials (biomaterials) for stopping bleeding and sealing wounds in surgery. Biomaterials are biologically compatible glues to seal surgical incisions, lubricants to help joint movement, and support on which living tissue is grown or shaped.

Exemplary modes of delivery include, but are not limited to, injection, insertion, implantation, or delivery within a scaffold that encapsulates the composition of the invention at the target tissue. In some embodiments, the composition is delivered to a natural or artificial cavity or chamber of a tooth of a subject by injection. When the compositions of the invention are dissolved in a solution, they can be injected into the tissue by a syringe.

The present invention also includes methods for removing tough gel adhesives (any tough gel adhesives and not limited to the tough gel adhesives described in the present disclosure) from a tissue surface without damaging the tissue surface. In particular, the present invention discloses a biocompatible and convenient method to detach tough gel adhesives on-demand. The method includes the steps of a) treating the tough gel with a removal solution; b) exposing the tough gel to the removal solution for about 1-100 minutes; and c) removing the tough gel adhesive from the tissue surface.

In some embodiments, the removal solution effectively weakens the interpenetrating network (IPN) of the tough gel or the covalent interaction of the adhesive layer. In one embodiment, the removal solution comprises a substance selected from the group consisting of ethanol, citric acid, hydrogen peroxide, alginate lyase, and lysozyme, or a combination thereof. In one embodiment, the removal solution comprises about 40-90% v/v ethanol, about 1-50 mM EDTA, about 20-70 mM citric acid, about 20-50% w/w hydrogen peroxide, about 1.0 mg/ml to about 10 mg/ml of alginate lyase, and/or about 10 mg/ml to 100 mg/ml of lysozyme. In one embodiment, the removal solution comprises about 40%, 50%, 60%, 70%, 80% or 90% v/v ethanol. In one embodiment, the removal solution comprises about 1 mM, 3 mM, 5 mM, 10 mM, 15 mM, 20 mM, 25 mM, 30 mM, 35 mM, 40 mM or 50 mM EDTA. In one embodiment, the removal solution comprises about 20 mM, 30 mM, 40 mM, 50 mM, 60 mM or 70 mM citric acid. In one embodiment, the removal solution comprises about 20%, 25%, 30%, 35%, 40%, 45% or 50% w/w hydrogen peroxide. In one embodiment, the removal solution comprises alginate lyase at about 1.0 mg/ml, 1.5 mg/ml, 2.0 mg/ml, 2.5 mg/ml, 3.0 mg/ml, 3.5 mg/ml, 4.0 mg/ml, 4.5 mg/ml, 5.0 mg/ml, 6.0 mg/ml, 7.0 mg/ml, 8.0 mg/ml, 9.0 mg/ml, or 10.0 mg/ml. In one embodiment, the removal solution comprises lysozyme at about 10 mg/ml, 20 mg/ml, 25 mg/mlm 30 mg/ml, 40 mg/mlm 50 mg/ml, 60 mg/ml, 70 mg/ml, 75 mg/ml, 80 mg/ml, 90 mg/ml, or 100 mg/ml.

In one embodiment, treatment time with the removal solution ranges from about 1 minute to about 100 minutes, for example, about 1 minute, 2 minutes, 3 minutes, 5 minutes, 10 minutes, 15 minutes, 20 minutes, 30 minutes, 40 minutes, 50 minutes, 60 minutes, 70 minutes, 75 minutes, 80 minutes, 90 minutes, or 100 minutes. In a specific embodiment, treatment time with the removal solution is about 1 minute or about 10 minutes.

### IV. Kits

The present invention also provides kits. Such kits can include a biodegradable adhesive material described herein and, in certain embodiments, instructions for administration. Such kits can facilitate performance of the methods described herein. When supplied as a kit, the different components of the biodegradable adhesive material can be packaged in separate containers and admixed immediately before use. Components include, but are not limited to, a preformed biodegradable IPN hydrogel, a solution containing the high density primary amine component, and a coupling agent in solid form. In a particular embodiment, the present invention is directed to a three component system including a preformed biodegradable IPN alginate-based hydrogel; a dry powder mixture of EDC/NHS; and a aqueous solution of the high density primary amine polymer. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

In certain embodiments, kits can be supplied with instructional materials which describe performance of the methods of the invention. Detailed instructions may not be physically associated with the kit; instead, a user may be directed to an Internet web site specified by the manufacturer or distributor of the kit.

The present invention is further illustrated by the following examples, which are not intended to be limiting in any way. The entire contents of all references, patents and published patent applications cited throughout this application, as well as the Figures, are hereby incorporated herein by reference.

### EXAMPLES

### Example 1: Materials and Methods

The tough gels synthesized with different biodegradable covalent crosslinkers achieved maximum fracture toughness values greater than traditional non-degradable MBAA tough gels. Accelerated hydrolytic degradation studies suggests the degradation of PEGDA 250 and PEGDA 10 k tough gels in hydrolytic solution before 24 h. These results demonstrate that PEGDA can serve as a replacement for MBAA as the covalent crosslinker in the synthesis of tough gels without the need of major protocol changes or the sacrifice of any of the valuable MBAA tough gel properties. Allowing for the design of both degradable and tough hydrogels. The results obtained in this study provide a fundamental advance in the design of tough adhesive materials, permitting to further extend their utility in the biomedical field.

### Biodegradable Covalent Crosslinkers

PEG based covalent crosslinkers, for example PEGDA 250 and PEGDA 10 k, were obtained from commercial sources.

Synthesis of gelatin methacrylate (GelMA) crosslinkers Gelatin methacrylate (GelMA) was synthesized by allowing Type A porcine skin gelatin (commercially available) at 10% (w/v) to dissolve in stirred Dulbecco's phosphate buffered saline (DPBS) at 50° C. for 1 hour. Methacrylic anhydride (commercially available) was added dropwise to a final volume ratio of 1:4 methacrylic anhydride:gelatin solution. This resulted in GelMA with a degree of substitution of 80%. The solution was stirred at 50° C. for 1 hour, and then diluted 5× with DPBS. The resulting mixture was dialyzed in 12-14 kDa molecular weight cutoff tubing for 4 days against distilled water with frequent water replacement. The dialyzed solution was lyophilized, and the resulting GelMA was stored at −20° C. until use.

### Synthesis of Alginate Methacrylate (AlgMA) Crosslinkers

Using a procedure similar to the GelMA synthesis, AlgMA was synthesized. Alginate polymer was reacted with 2-aminoethyl methacrylate (AEMA) to obtain AlgMA.

### Synthesis of Oxidized Alginate Methacrylate (OxAlgMa) Crosslinkers

Methacrylated oxidized-alginate was prepared by reacting 200 mg of alginate-2.5% oxidized-(MVG, Nova matrix, Norway) with 2-Aminoethylmethacrylamide hydrochloride-AEME (Sigma-900652). 2.5% oxidized sodium alginate was dissolved in a 10 ml buffer solution [0.75% (wt/vol), pH ˜6.5] of 100 mM MES. The coupling reagents were added to activate the carboxylic acid groups of alginate (130 mg N-hydroxysuccinimide (NHS) and 280 mg 1-ethyl-3-(3-dimethylaminopropyl) carbodiimide hydrochloride (EDC)). After 5 min, AEMA (224 mg; molar ratio of NHS:EDC:AEMA=1:1.3:1.1) was added to the product and the solution was stirred at RT for 24 h. The mixture was precipitated in acetone, filtered, and dried in a vacuum overnight at RT.

Synthesis of Tough Gels with Different Biodegradable Covalent Crosslinkers

The tough adhesives combine a tough gel dissipative matrix and a bridging polymer with coupling reagents. Alginate (LF20/40 and 5 Mrad) and acrylamide were dissolved in Hank's balanced salt solution (HBSS) and stirred overnight at room temperature until completely homogeneous. This solution was then mixed with the biodegradable covalent crosslinker, N, N, N′, N′-Tetramethylethylenediamine (TEMED or TMEDA), calcium sulfate (CaSO₄H₂O) and ammonium persulfate (APS) and poured in a glass mold (80×15×1.5 mm) sealed with a glass cover. Finally, the mixture was left in the mold overnight at room temperature to ensure complete reaction. In one particular embodiment, tough gels were synthesized by combining a solution of 2% sodium alginate and 12% acrylamide in HBSS with certain covalent crosslikers, TEMED, ammonium persulfate, and calcium sulfate dehydrate

### Tough Adhesive Preparation

Chitosan was dissolved in ddH₂O at 4% w/w and combined with 1-ethyl-3-(3-dimethylaminopropyl) carbodiimide (EDC) and sulfated N-hydroxysuccinimide (NHS) as coupling reagents (12 mg/ml). The adhesive (˜300 μl) was applied to the surface of the tough gel (15×1.5×40 mm²) before contacting with the tissue surface and applying compression for 45-60 min.

### Mechanical Testing

A mechanical testing setup (Instron, Norwood, Mass.) was used for tensile tests. For tensile testing, a rectangular strip of the tough gel (25×15×1.5 mm³) was glued between two pieces of sand paper on each side. For fracture energy testing, a rectangular strip of the tough gel (15×40×1.5 mm³) was glued to two rectangular acrylic pieces on each side and cut using a razor blade in the middle of the sample gauge section, with the intention of creating a horizontal edge crack 20 mm of length. The stretch rate for tensile testing was 100 mm/min, and for fracture energy testing it was 20 mm/min. Force and extension were recorded by the Instron machine (model 3342 with load cell of maximum 10 N) at 50 Hz throughout the test. From the stress-stretch curves, the matrix maximum stretch, maximum stress, and toughness were calculated.

### Adhesion Energy Measurements

Adhesion energy was measured with peeling tests using a mechanical testing setup (Instron, Norwood, Mass.) under uniaxial tension (100 mm/min). The tough adhesive was bonded to a thin plastic film on one side and adhered to the tissue on the other side. Adhesion energy was calculated by multiplying the maximum value of force and width ratio times two.

### In Vitro Hydrolytic or Enzymatic Degradation of Tough Gels

Preliminary experiments to assay the degree of biodegradability of the tough gels with covalent crosslinkers were done. The experiments were carried out by incubating MBAA and PEGDA tough gels in an accelerated hydrolytic solution for 6 days. PEGDA 250 and PEGDA 10 k tough gels where able to pass through a 30 G needle within 24 h in solution, which suggest the degradation of the tough gels. However, traditional MBAA tough gels remained stable during the period of 6 days in the hydrolytic solution.

Tough gel degradation was evaluated over time by placing hydrogels in hydrolytically degrading or enzymatically degrading buffers. For accelerated hydrolytic degradation, PEGDA and MBAA tough gel disks (8 mm in diameter) were incubated in a 5 mM sodium hydroxide (NaOH) for 24, 48, 72, 96, 120, and 144 h with daily solution changes. Swelling ratio was monitored daily and calculated at each time point relative to initial dry mass. The percent degradation was calculated by the dried weight after digestion divided by the weight of untreated tough gels (n=3/group). The same procedure was followed for PEGDA tough gels hydrolytic degradation studies using a 0.1 mM NaOH solution.

Alternatively, three circular gels (6 mm diameter, 1.5 mm thickness) were incubated in a 10 mM sodium hydroxide (NaOH) solution with 1.5 mM calcium chloride at 37° C. with daily solution changes for six days. Samples were collected daily rinsed with deionized water and freeze dried to monitor total weight change.

For accelerated enzymatic degradation, GelMA crosslinked gels were incubated in HBSS buffer spiked with 1.5 mM calcium chloride with 25 U/ml Collagenese II at 37° C. with daily solution changes. Samples were collected daily rinsed with DI water and freeze-dried to monitor total weight change.

### Fracture Energy

To determine the optimal concentration of covalent crosslinker in tough gels for maximum fracture toughness, in a series of experiments, the ratio of short chain and long chain alginates was fixed to 1:1, and the percent weight concentration of the covalent crosslinkers was varied. The evaluated concentrations were chosen starting from optimal covalent crosslinker concentration used in traditional MBAA tough gels (J. Y. Sun et al., “Highly stretchable and tough hydrogels,” Nature, vol. 489, no. 7414, pp. 133-136, 2012). Thereafter, the fracture energy of the tough gels with different covalent crosslinkers was measured by performing tensile tests on notched and unnotched samples following the pure shear test procedure (X. Zhao, “Multi-scale multi-mechanism design of tough hydrogels: Building dissipation into stretchy networks,” Soft Matter, vol. 10, no. 5, pp. 672-687, 2014).

### High Frequency Ultrasound Imaging (HFUS)

High frequency ultrasound (HFUS) (VisualSonics Vevo 770 and Vevo 3100; 35-50 MHz) was used to evaluate gel swelling and degradation in vivo. Axial images (30-40 μm resolution) were acquired that captured the skin and hydrogel. Images were quantified for the thickness of the hydrogel and surrounding capsule. For tough gels crosslinked with MBAA, PEGDA 250, and poloxamer diacrylate (Polox DA), imaging was completed after 1, 2, 4, and 8 weeks post implantation. For tough gels cross linked with MBAA, GelMA, HAMA, and OxAlgMA, imaging was completed after 4, 8, and 16 weeks. Images were analyzed for hydrogel thickness using ImageJ (NIH).

GPC Analysis of Chitosan Degradation with Lysozyme

Gel permeation chromatography (GPC) analysis was performed with Viscotek TDAmax is equipped with a GPCmax solvent and sample delivery module, a TDA 305 triple detector, a UV detector 2600, a solvent saver device, and OmniSec software, the GPC column was single G4000PW×1 (Tosoh Bioscience) with flow rate 0.75 ml/min mobile phase-0.1M sodium nitrate (NaNO₃), 0.01 M monosodium phosphate NaH₂PO₄ and 0.075% sodium azide (NaN₃), buffered to pH 3.0 with phosphoric acid. Three times filtered through 0.1 m PES filter and sample injection volume was 100 μL.

### Subcutaneous Injury Model

Balb/C mice at 6-8 weeks of age had tough hydrogels implanted subcutaneous (IACUC approved). Briefly, animals were anesthetized with isoflurane (2-2.5%) and given buprenorphine (0.5 mg/kg) for pain management. Hair on the mouse dorsum was removed with clippers and depilatory cream prior to adding three separate washes of betadine and ethanol. Animals were then transferred to the sterile field and placed beneath a separate sterile fenestrated drape. A small 6 mm incision was made through skin in the animal's back perpendicular to its midline and a pocked was created using scissors. Four separate gels (D=3 mm, th=1.5 mm) were then implanted subcutaneously and the skin was closed with 4-0 Vicryl suture. Animals were monitored daily and evaluated for subsequent assays.

### Example 2: Comparison of Mechanical Properties of Tough Gel Adhesives Having Biodegradable Covalent Crosslinkers or Non-Biodegradable Covalent Crosslinkers

### Fracture Energy

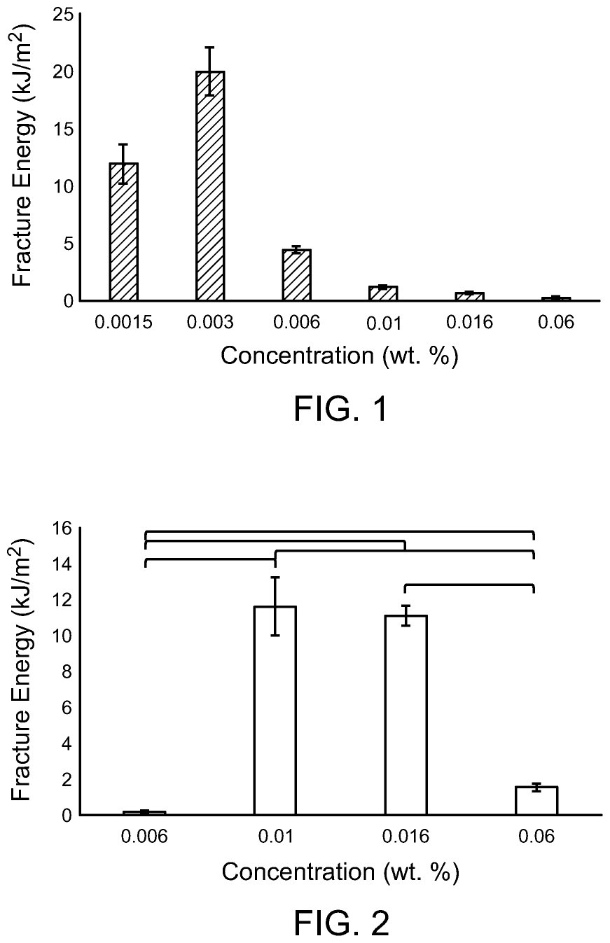

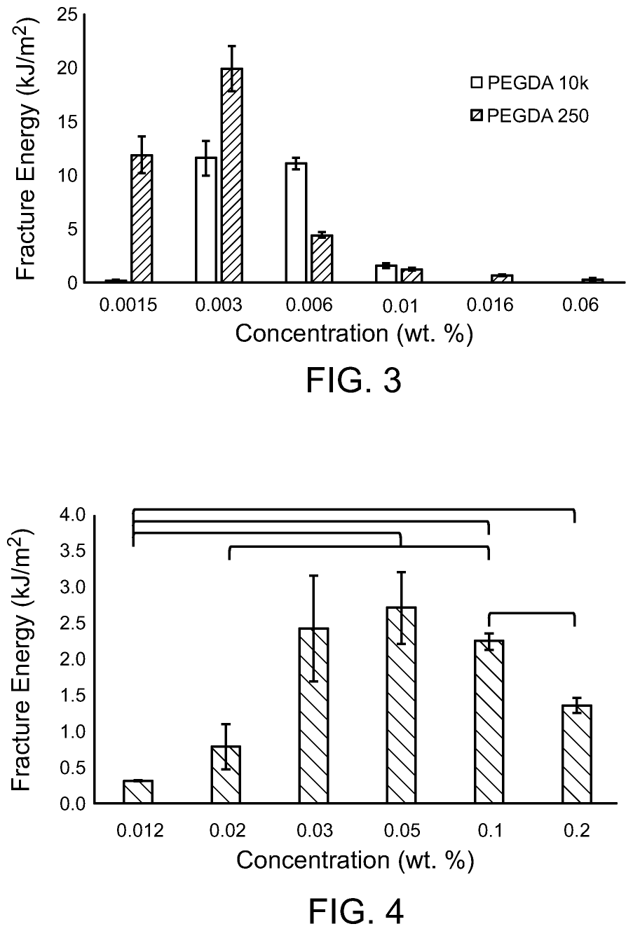

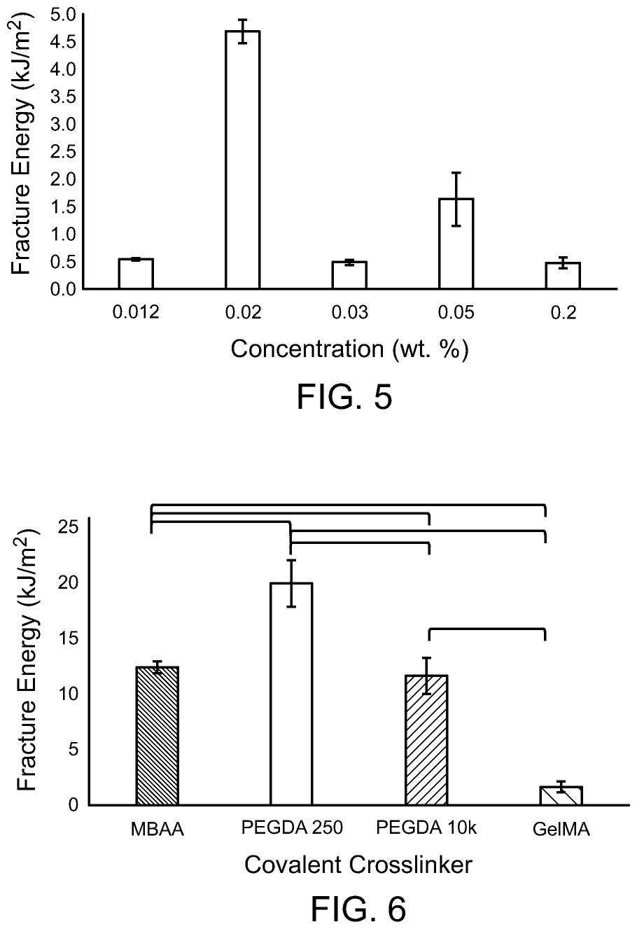

Results show that for PEGDA 250 tough gels reached a critical stretch at rupture maximum at 0.003% w/w with a fracture energy value of ˜20 kJ/m² (FIG. 1). Tough gels with PEGDA 10 k as covalent crosslinker exhibited a maximum fracture toughness value of about 10 kJ/m², at a covalent crosslinker concentration of 0.01 and 0.016% w/w, where no statistical difference was found between the values obtained for both percent weight concentrations (FIG. 2), as compared to 20 kJ/m² fracture energy value for PEGDA 250 tough gels (FIG. 3). As shown in FIG. 4 and FIG. 5, GelMA and AlgMA-5Mard tough gels exhibited low fracture toughness values of ˜2.5 kJ/m² and ˜4.5 kJ/m², respectively. FIG. 6 shows the fracture toughness values for the best performing percent weight concentration of each covalent crosslinker in the tough gels. A maximum fracture energy value of ˜20 kJ/m² was achieved for PEGDA 250 tough gels. This value is 1.7 times higher than that of traditional non-degradable tough gels. These results demonstrate that the covalent crosslinker used for tough gel synthesis and its concentration strongly affects the properties of alginate-polyacrylamide tough gels. Moreover, it shows that PEGDA 250 and PEGDA 10 k can be used to replace MBAA as the covalent crosslinker in the synthesis of tough gels.

### Maximum Stress, Stretch and Toughness

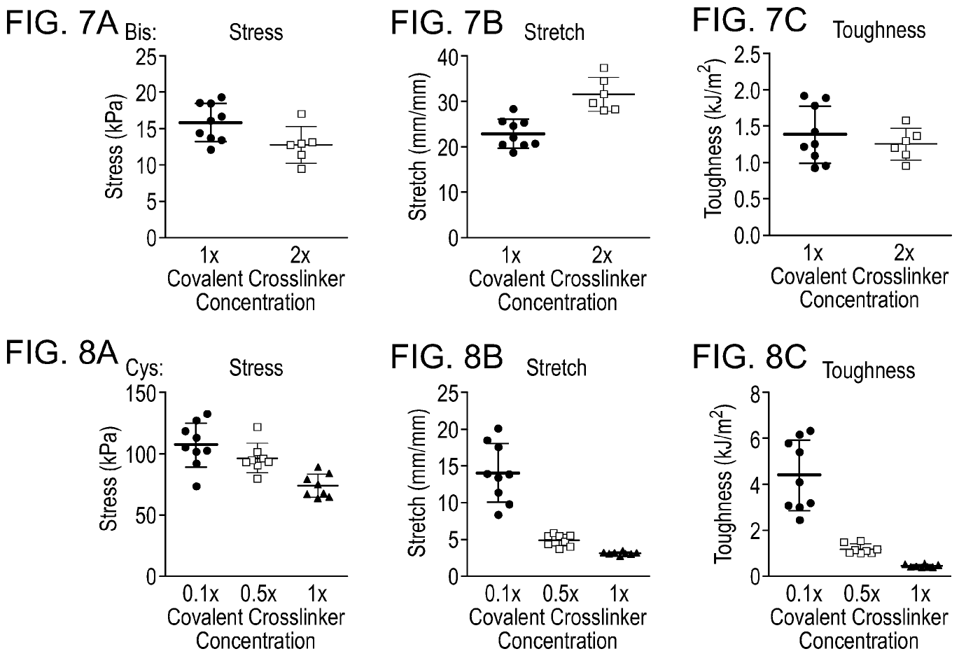

Referring to FIGS. 7A through 14C, all hydrogels incorporating hydrolyzable crosslinkers (i.e., PEGDA 250, Polox DA, Bis, OxAlgMA), enzymatically cleavable crosslinkers (GelMA, HAMA) or reduction-cleavable crosslinkers (Cys) demonstrated maximum stretch, stress, and toughness superior to traditional hydrogel systems made with non-biodegradable crosslinkers when tested in tension. In these figures, 16 mg of crosslinker in 10 ml of buffer is equivalent to 1× concentration, or 0.01 wt %).

When comparing different covalent crosslinker concentrations for Bis, 2× performed best overall for max stress, stretch, and toughness (FIGS. 7A-7C).

When comparing different covalent crosslinker concentrations for Cys, the 0.1× concentration performed best overall for max stress, stretch, and toughness (FIGS. 8A-8C).

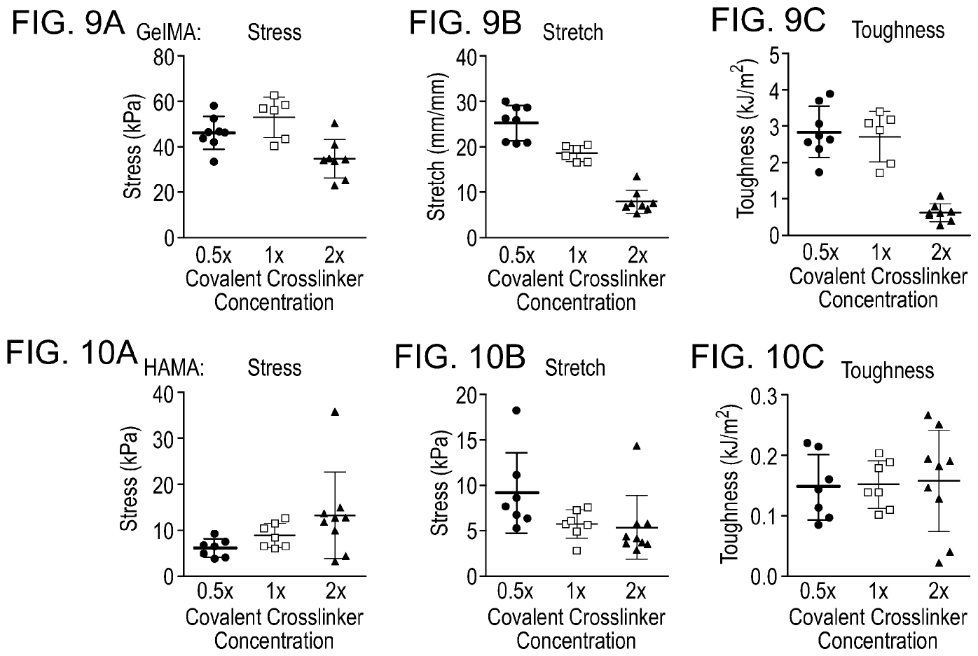

When comparing different covalent crosslinker concentrations for GeIMA, the 0.5× concentration performed best overall for max stress, stretch, and toughness (FIGS. 9A-9C).

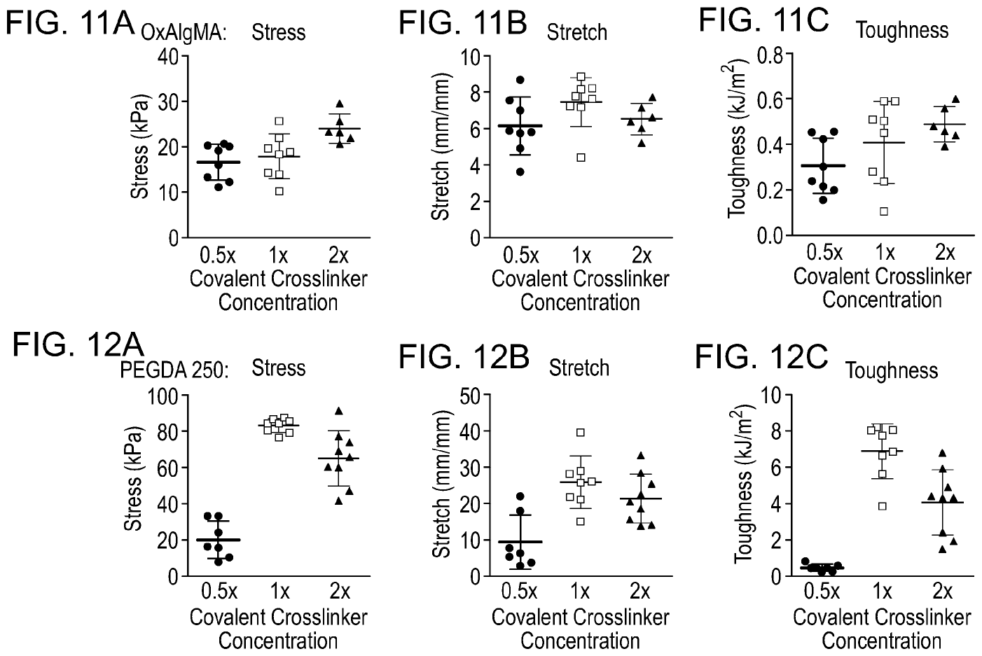

When comparing different covalent crosslinker concentrations for HAMA, the 1× concentration performed best overall for max stress, stretch, and toughness (FIGS. 10A-10C).

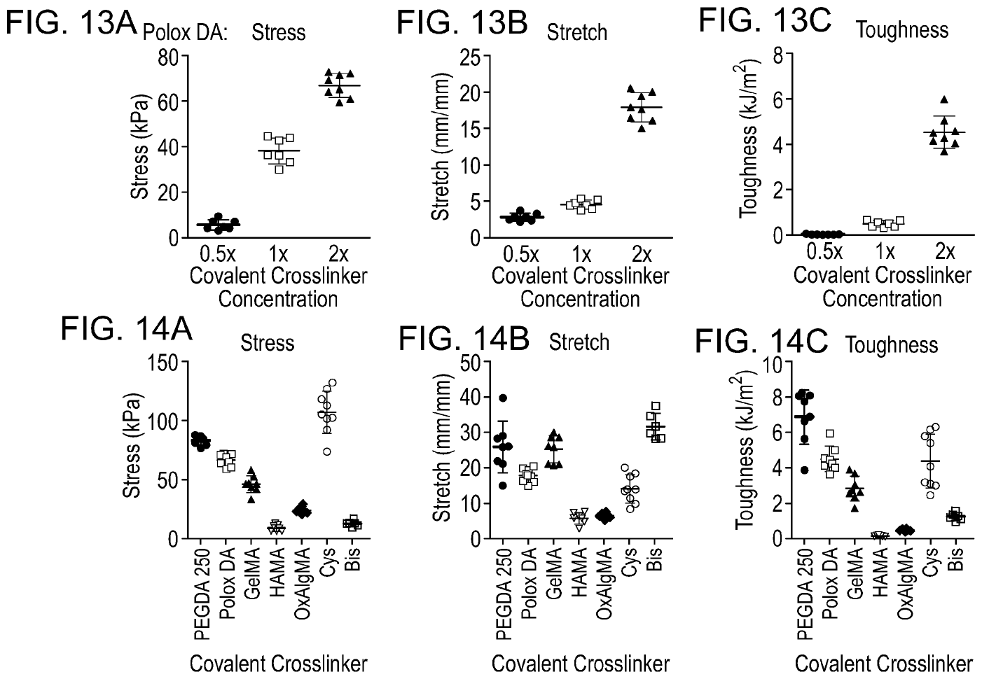

When comparing different covalent crosslinker concentrations for OxAlgMA, the 1× concentration performed best overall for max stress, stretch, and toughness (FIGS. 11A-11C).

When comparing different covalent crosslinker concentrations for PEGDA 250, the 1× concentration performed best overall for max stress, stretch, and toughness (FIGS. 12A-12C).

When comparing different covalent crosslinker concentrations for Polox DA, the 2× concentration performed best overall for max stress, stretch, and toughness (FIGS. 13A-13C).

When comparing across crosslinkers, PEGDA 250 and Cys achieved the best maximum stresses (>75 kPa); PEGDA 250, GeIMA, and Bis had the best maximum stretches (˜25 mm/mm); and PEGDA 250 had the highest toughness (7 kJ/m²) (FIGS. 14A-14C).

### Example 3: Comparison of In Vitro and In Vivo Degradation Rates of Tough Gel Adhesives Having Biodegradable Covalent Crosslinkers or Non-Biodegradable Covalent Crosslinkers

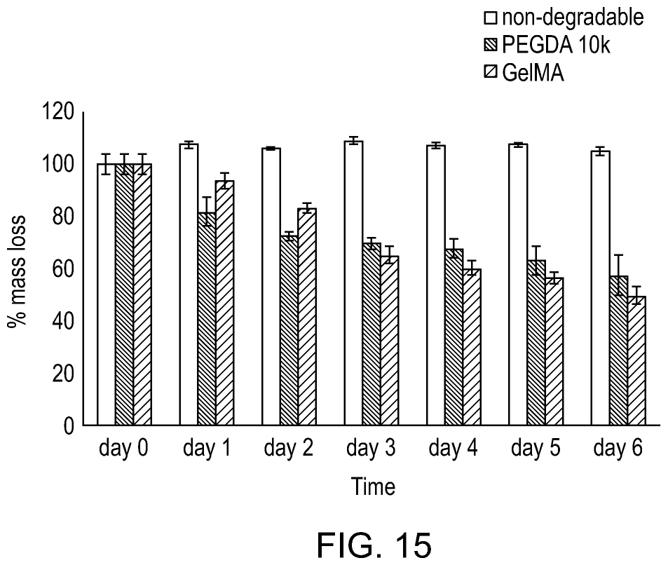

Referring to FIG. 15, it was observed that over time, non-degradable gels crosslinked with MBAA demonstrated no change in mass loss through day 6. In contrast, PEGDA 10 k and GelMA gels exhibited a precipitous decline in mass loss through day 6. It is noted that the rate of mass loss for GeIMA crosslinked gels was dependent on the amount of collagenase II enzyme added (data not shown).

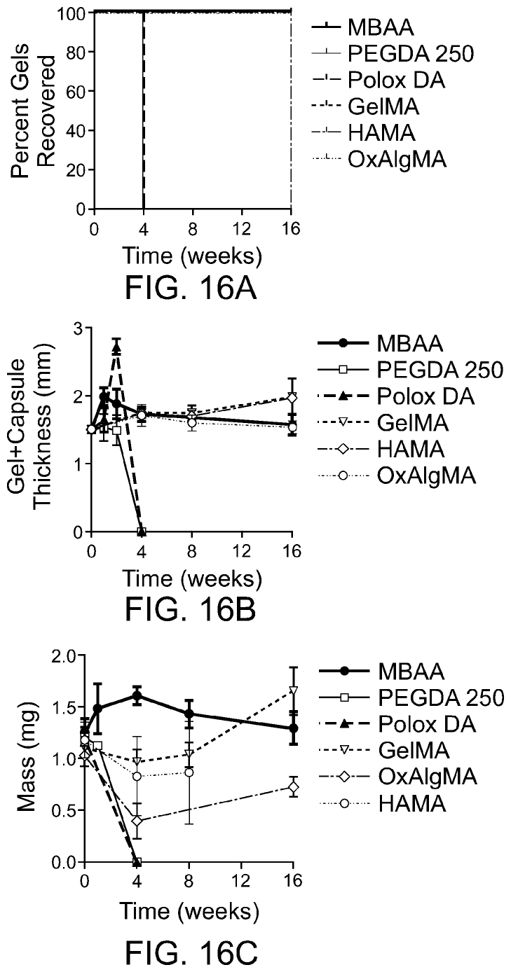

Following subcutaneous implantation, control gels made by using non-degradable MBAA crosslinkers did not degrade, as hypothesized. The hydrolyable crosslinkers PEGDA 250 and Polox DA degraded rapidly within 4 weeks. HAMA, GelMA, and OxAlgMA crosslinked gels had slower degradation after subcutaneous implantation, with all gels present through 8-week. GelMA and OxAlgMA crosslinked gels were present through 16 weeks (FIG. 16A). In agreement with gross observation at euthanasia, hydrogel dry weights and gel thicknesses decreased by 4 weeks for PEGDA and Polox DA hydrogels. In contrast, dry weights and gel thickness were maintained for GelMA and OxAlgMA through the duration of the study (FIGS. 16B and 16C).

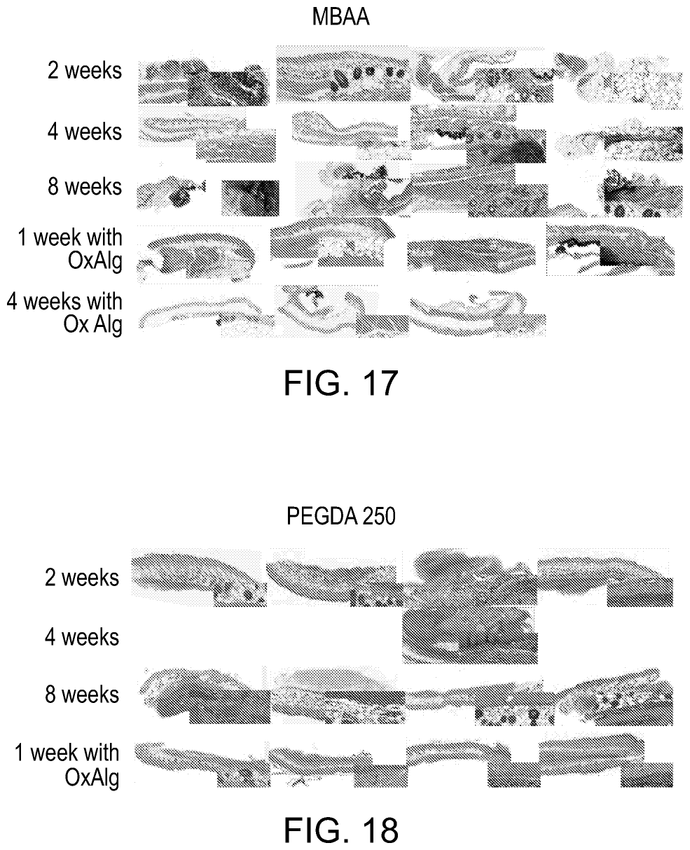

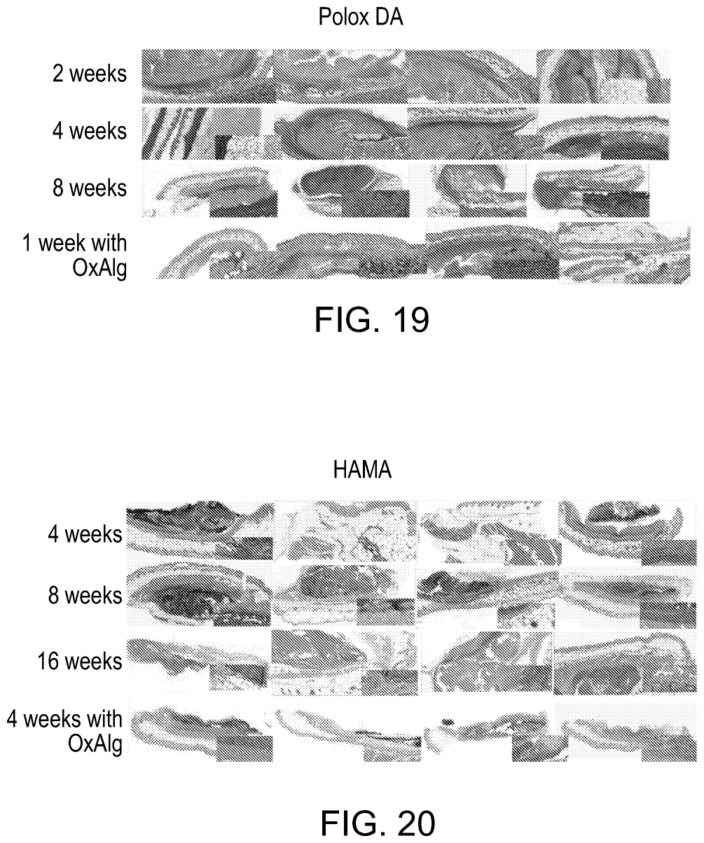

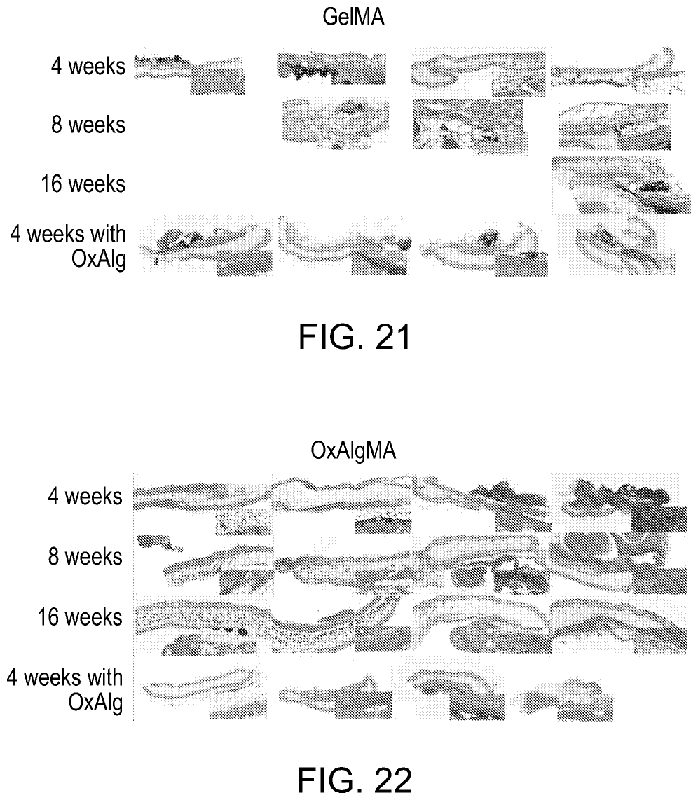

Subcutaneous implantation of the tough gels was further evaluated for histology after 1, 2, 4, 8, or 16 weeks. These degradable crosslinkers included PEGDA 250 (FIG. 18), poloxamer diacrylate (Polox DA) (FIG. 19), HAMA (FIG. 20), GelMA (FIG. 21), and OxAlgMA (FIG. 22). The nondegradable crosslinker MBAA was used as a control (FIG. 17). In some instances, the ionically crosslinked alginate network was made degradable by substituting for oxidized alginate. Consistent with the dry weight measurements over time, PEGDA 250 and Polox DA crosslinked gels were not detectable after 4 weeks post implantation. Similarly, HAMA crosslinked gels were not detectable after 16 weeks post implantation. Overall, the biocompatibility of samples was positive and similar to MBAA hydrogels.

### Example 4: Removal of Tough Gel Adhesives

Tough gel adhesives have demonstrated unprecedented adhesion energies to wet and moving tissue surfaces, and excellent biocompatibility. The tough gel adhesive is able to achieve high adhesion energies through a two-layer structure, a dissipative matrix (tough gel) and a positively charged adhesive layer that interacts electrostatically and forms covalent bonds with the tough gel and the tissue surfaces. Although strong adhesion is generated, for many indications it is necessary to remove the tough gel adhesive on demand. The objective was to develop an on-demand, easy to use and biocompatible detachment strategy for the tough gel adhesive. This was achieved by treating the tough gel with a solution that weakens the dissipative matrix.

### Tough Gel Synthesis

Alginate and acrylamide were dissolved in HBSS without calcium and magnesium overnight. This solution was then mixed with TEMED, calcium sulfate and ammonium persulfate, and poured in a glass mold sealed with a glass cover.

### Treatment of Tough Gels in Different Solutions

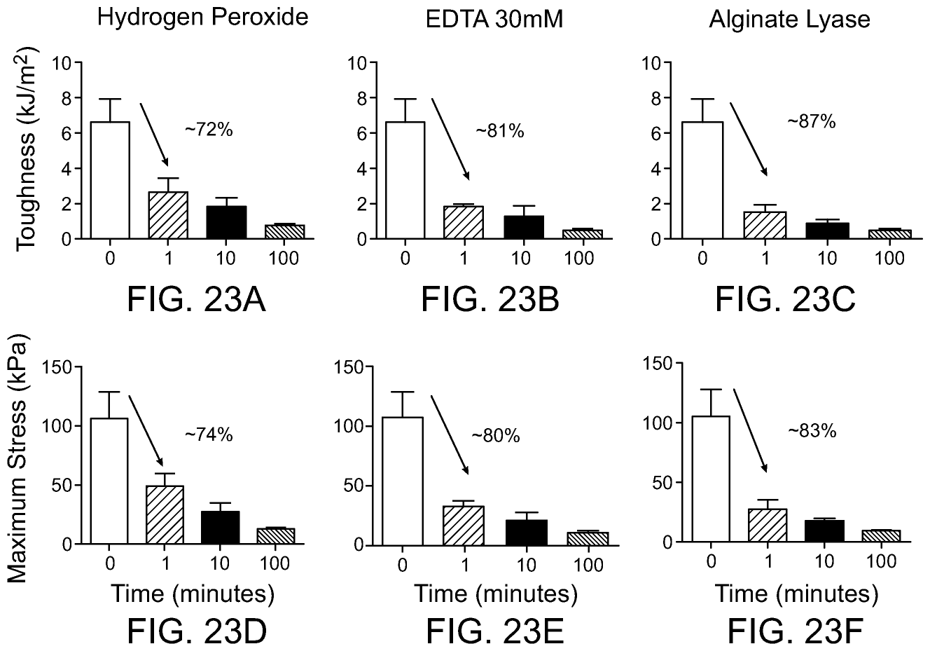

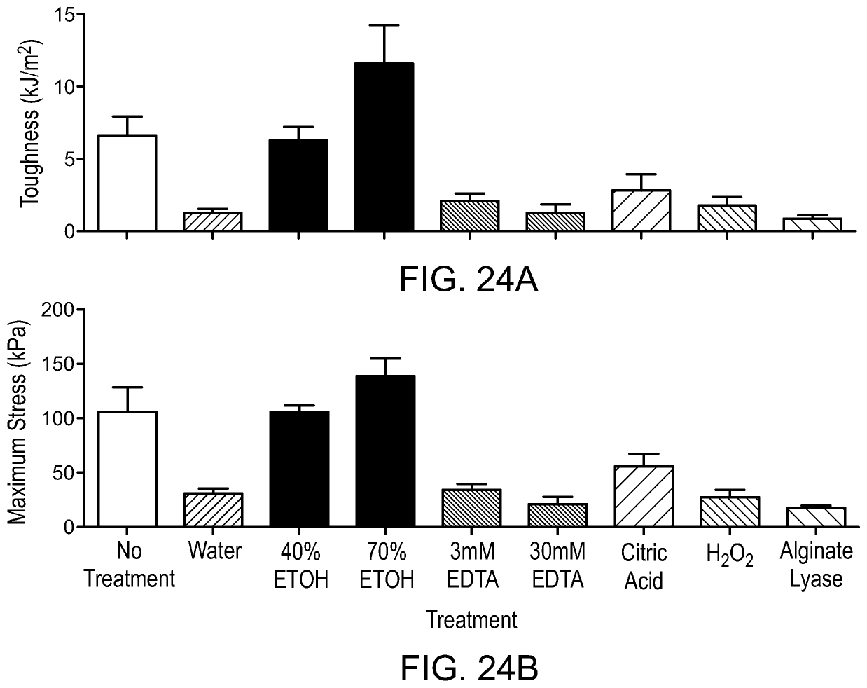

To promote degradation, tough gels were submerged in water, ethanol (40 and 70%), citric acid (50 mM), EDTA (3 and 30 mM), hydrogen peroxide (35 wt %), or alginate lyase for 1, 10 and 100 min (FIGS. 23A-23F, 24A and 24B). Gels were then removed from solution and prepared for mechanical tensile testing.

Alternatively, 3 mg/ml chitosan (54046; 90% deacetylated) solutions were incubated with solutions of lysozyme, at increasing concentrations of 17 mg/ml, 37 mg/ml and 75 mg/ml of lysozyme solutions, and at increasing incubation times of 1 min, 10 min, 30 min and 100 min. The lysozyme used was L6876 from chicken egg white protein ≥90%, ≥40,000 units/mg from sigma. The highest concentration of lysozyme (75 mg/ml) resulted in the highest decrease in weight average molecular weight from 280 kD to 178 kD. The change in the chitosan weight (weight average (Mw) and number average (Mn) molecular weight) as resulted from 75 mg/mL lysozyme degradation over 100 min is summarized in Table 1.

TABLE 1

| Time | Chitosan | | Chitosan + lysozyme (75 mg/ml) | |
|---|---|---|---|---|
| Min | Mw (Da) | Mn (Da) | Mw (Da) | Mn (Da) |
| 1 | 283,104 | 125,007 | 225,041 | 97,596 |
| 10 | 277,776 | 121,468 | 196,743 | 96,280 |
| 30 | 274,913 | 115,328 | 178,318 | 87,063 |
| 100 | 272,745 | 118,436 | 177,527 | 90,904 |

Additionally, 3 mg/mL chitosan (54046; 90% deacetylated; 54039 85% deacetylated) solutions incubated with solutions of 75 mg/ml or 150 mg/ml lysozyme. The lysozyme used was L6876 from chicken egg white protein ≥90%, ≥40,000 units/mg from sigma. The Mw decreases were similar between these two samples incubated with 75 mg/ml or 150 mg/ml lysozyme, and also between two chitosan samples at different levels of deacylation. The change in the chitosan weight (weight average (Mw) and number average (Mn) molecular weight) as resulted from lysozyme degradation over 100 min is summarized in Tables 2 and 3.

TABLE 2

| | Chitosan (54046) + | | Chitosan (54046) + | |
|---|---|---|---|---|
| Time | lysozyme (75 mg/ml) | | lysozyme (150 mg/ml) | |
| Min | Mw (Da) | Mn (Da) | Mw (Da) | Mn (Da) |
| 0 | 226,690 | 123,294 | 226,690 | 123,294 |
| 100 | 114,260 | 64,920 | 116,845 | 62,947 |

TABLE 3

| | Chitosan (54039) + | | Chitosan (54039) + | |
|---|---|---|---|---|
| Time | lysozyme (75 mg/ml) | | lysozyme (150 mg/ml) | |
| Min | Mw (Da) | Mn (Da) | Mw (Da) | Mn (Da) |
| 0 | 45,307 | 28,049 | 45,307 | 28,049 |
| 100 | 27,089 | 18,192 | 28,798 | 22,410 |

### Tensile Testing

Tough gel strips (15×15×1.5 mm³) were glued between two pieces of acrylic. A mechanical testing setup (Instron, Norwood, Mass.) was used to evaluate tensile mechanical properties (rate: 100 mm/min). Recorded force and extension data were used to compute the toughness, maximum stress, and maximum stretch. One-way ANOVA with post hoc T-tests with Bonferroni corrections were used to evaluate the effect of chemical treatment and time on hydrogel mechanical properties.

Decrease in Tensile Mechanical Properties after Treatment

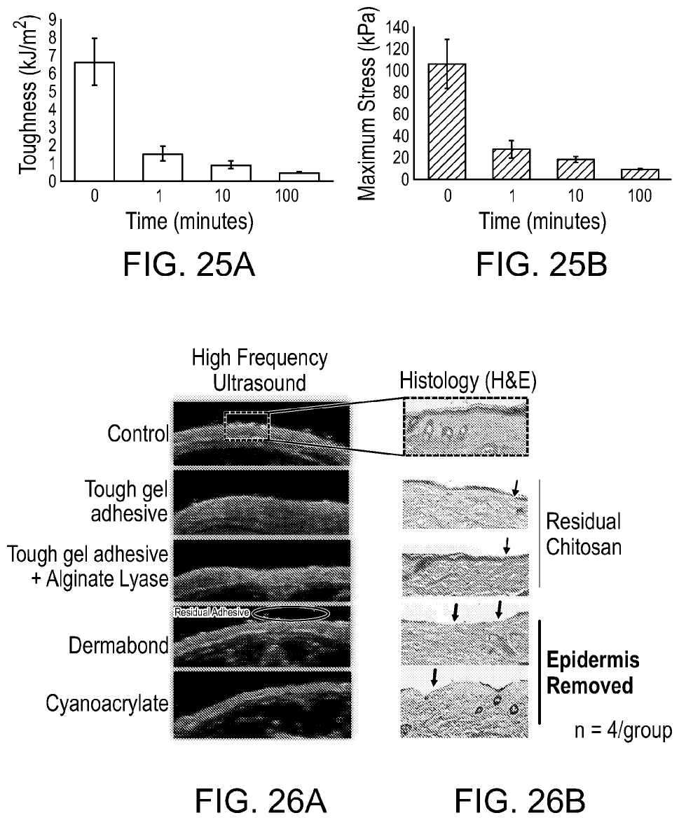

The tensile mechanical properties of tough gels demonstrated significant changes after being treated with many solutions (i.e., water, EDTA, citric acid, EDTA, hydrogen peroxide and alginate lyase). Decreases in gel mechanical properties were observed within 1-minute of treatment (FIGS. 23A-23F, 24A and 24B). In particular, tough gels treated with alginate lyase demonstrated a dramatic decrease (˜77%) in toughness and maximum stress (˜74%) after 1 minute (FIGS. 25A and 25B). Short-term exposure to the solution was demonstrated to greatly affect tough gel tensile mechanical properties.

Histology Analysis after Treatment

The commercially available adhesives and the tough gel adhesive were applied to back of mice and peeled to examine the effects of adhesive removal on the tissue surface (skin). For comparison, a detailed microscale skin evaluation was performed following adhesive removal (FIGS. 26A and 26B). As can be seen from the histology analysis (FIGS. 26A and 26B), the tough gel adhesive removal with or without treatment with alginate lyase showed no damage to the epidermis, whereas the commercially available adhesives damaged the tissue surface (epidermis).

### EQUIVALENTS

Those skilled in the art will recognize, or be able to ascertain using no more than routine experimentation, many equivalents to the specific embodiments of the invention described herein. such equivalents are intended to be encompassed by the following claims. The contents of all references, patents and published patent applications cited throughout this application are incorporated herein by reference.

1. A composition comprising a biodegradable interpenetrating networks (IPN) hydrogel, comprising a first polymer network and a second polymer network, wherein the first polymer network comprises a first polymer covalently crosslinked with a biodegradable covalent crosslinker and the second polymer network comprises a second polymer crosslinked with ionic or physical crosslinks.

2. A composition comprising a biodegradable tough adhesive material, comprising
a) an IPN hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks;
b) an adhesive bridging polymer; and
c) a coupling agent.

3. The composition of claim 1 or claim 2, wherein the first polymer is selected from the group consisting of polyacrylamide, poly(hydroxyethylmethacrylate) (PHEMA), poly(vinyl alcohol) (PVA), polyethylene glycol (PEG), polyphosphazene, collagen, gelatin, poly(acrylate), poly(methacrylate), poly(methacrylamide), poly(acrylic acid), poly(N-isopropylacrylamide) (PNIPAM), poly(N,N-dimentylacrylamide), poly(allylamine) and copolymers thereof.

4. The composition of claim 3, wherein the first polymer comprises polyacrylamide.

5. The composition of any one of claims 1 to 4, wherein the biodegradable covalent crosslinker is selected from the group consisting of a poly(ethylene glycol) acrylate, a gelatin acrylate, a hyaluronic acid acrylate, an alginate acrylate, a poloxamer acrylate, a disulfide-based crosslinker.

6. The composition of any one of claims 1 to 5, wherein the biodegradable covalent crosslinker is selected from the group consisting of a poly(ethylene glycol) acrylate, a gelatin acrylate, a hyaluronic acid acrylate and an alginate acrylate.

7. The composition of claim 5, wherein the biodegradable covalent crosslinker is selected from the group consisting of a poly(ethylene glycol) diacrylate (PEGDA), gelatin methacrylate (GelMA), alginate methacrylate (AlgMA), hyaluronic acid methacrylate (HAMA), a poloxamer acrylate, a disulfide-based acrylate, and N,N′-bis(acryloyl)cystamine (Cys).

8. The composition of claim 7, wherein the biodegradable covalent crosslinker is selected from the group consisting of poly(ethylene glycol) diacrylate 250 (PEGDA 250), gelatin methacrylate (GeIMA), hyaluronic acid methacrylate (HAMA), oxidized alginate methacrylate (OxAlgMA), poloxamer diacrylate (Polox DA), bis(2-methacryloyl)oxyethyl disulfide (Bis), and N,N′-bis(acryloyl)cystamine (Cys).

9. The composition of claim 6, wherein the biodegradable covalent crosslinker is selected from the group consisting of a poly(ethylene glycol) diacrylate (PEGDA), a gelatin methacrylate (GelMA) and a methacrylated alginate (AlgMA).

10. The composition of any one of claims 1-9, wherein the biodegradable covalent crosslinker has a molecular weight of about 100 Da to about 40,000 Da.

11. The composition of claim 10, wherein the biodegradable covalent crosslinker has a molecular weight of about 250 Da to about 20,000 Da.

12. The composition of claim 7, wherein the concentration of the poly(ethylene glycol) diacrylate (PEGDA) in the hydrogel is about 0.001 wt. % to 0.05 wt. % based on the weight of the first polymer.

13. The composition of claim 7, wherein the concentration of the poloxamer acrylate in the hydrogel is about 0.001 wt. % to 0.05 wt. % based on the weight of the first polymer.

14. The composition of claim 7, wherein the concentration of the gelatin methacrylate (GeIMA) in the hydrogel is about 0.001 wt. % to 0.05 wt. % based on the weight of the first polymer.

15. The composition of claim 8, wherein the concentration of the oxidized alginate methacrylate (OxAlgMA) in the hydrogel is about 0.001 wt. % to 0.05 wt. % based on the weight of the first polymer.

16. The composition of claim 7, wherein the concentration of the hyaluronic acid methacrylate (HAMA) in the hydrogel is about 0.001 wt. % to 0.05 wt. % based on the weight of the first polymer.

17. The composition of claim 7, wherein the concentration of the disulfide-based acrylate in the hydrogel is about 0.005 wt. % to 0.03 wt. % based on the weight of the first polymer.

18. The composition of claim 7, wherein the concentration of N,N′-bis(acryloyl)cystamine (Cys) in the hydrogel is about 0.0005 wt. % to 0.01 wt. % based on the weight of the first polymer.

19. The composition of any one of claims 1 to 18, wherein the second polymer is selected from the group consisting of alginate, pectate, carboxymethyl cellulose, oxidized carboxymethyl cellulose, hyaluronate, chitosan, κ-carrageenan, ι-carrageenan and λ-carrageenan, wherein the alginate, carboxymethyl cellulose, hyaluronate chitosan, κ-carrageenan, ι-carrageenan and λ-carrageenan are each optionally oxidized, wherein the alginate, carboxymethyl cellulose, hyaluronate chitosan, κ-carrageenan, ι-carrageenan and λ-carrageenan optionally include one or more groups selected from the group consisting of methacrylate, acrylate, acrylamide, methacrylamide, thiol, hydrazine, tetrazine, norbornene, transcyclooctene and cyclooctyne.

20. The composition of claim 19, wherein the second polymer comprises alginate.

21. The composition of claim 20, wherein the alginate is oxidized alginate.

22. The composition of claim 20 or claim 21, wherein the alginate is comprises a mixture of a high molecular weight alginate and a low molecular weight alginate.

23. The composition of claim 22, wherein the ratio of the high molecular weight alginate to the low molecular weight alginate is about 5:1 to about 1:5.

24. The composition of any one of claims 1 to 23, wherein the first polymer network and the second polymer network are covalently coupled.

25. The composition of any one of claims 2 to 24, wherein the adhesive bridging polymer is a high density primary amine polymer.

26. The composition of claim 25 wherein the high density primary amine polymer is selected from the group consisting of chitosan, gelatin, collagen, polyallylamine, polylysine, and polyethylenimine.

27. The composition of claim 26, wherein the high density primary amine polymer is chitosan.

28. The composition of any one of claims 2 to 27, wherein the coupling agent includes a first carboxyl activating agent.

29. The composition of claim 28, wherein the first carboxyl activating agent is a carbodiimide.

30. The composition of claim 29, wherein the carbodiimide is selected from the group consisting of 1-ethyl-3-(3-dimethylaminopropyl)carbodiimide (EDC, EDAC or EDCI), dicyclohexylcarbodiimide (DCC) and diisopropylcarbodiimide (DIC).

31. The composition of any one of claims 2 to 30, wherein the coupling agent further includes a second carboxyl activating agent.

32. The composition of claim 31, wherein the second carboxyl activating agent is N-hydroxysuccinimide (NHS), N-hydroxysulfosuccinimide (sulfo-NHS), hydroxybenzotriazole (HOBt), dimethylaminopyridine (DMAP), Hydroxy-3,4-dihydro-4-oxo-1,2,3-benzotriazine (HOOBt/HODhbt), 1-Hydroxy-7-aza-1H-benzotriazole (HOAt), Ethyl 2-cyano-2-(hydroximino)acetate, Benzotriazol-1-yloxy-tris(dimethylamino)-phosphonium hexafluorophosphate (BOP), Benzotriazol-1-yloxy-tripyrrolidino-phosphonium hexafluorophosphate, 7-Aza-benzotriazol-1-yloxy-tripyrrolidinophosphonium hexafluorophosphate), Ethyl cyano(hydroxyimino)acetato-O2)-tri-(1-pyrrolidinyl)-phosphonium hexafluorophosphate, 3-(Diethoxy-phosphoryloxy)-1,2,3-benzo[d] triazin-4(3H)-one, 2-(1H-Benzotriazol-1-yl)-N,N,N′,N′-tetramethylaminium tetrafluoroborate/hexafluorophosphate, 2-(6-Chloro-1H-benzotriazol-1-yl)-N,N,N′,N′-tetramethylaminium hexafluorophosphate), N-[(5-Chloro-1H-benzotriazol-1-yl)-dimethylamino-morpholino]-uronium hexafluorophosphate N-oxide, 2-(7-Aza-1H-benzotriazol-1-yl)-N,N,N′,N′-tetramethylaminium hexafluorophosphate, 1-[1-(Cyano-2-ethoxy-2-oxoethylideneaminooxy)-dimethylamino-morpholino]-uronium hexafluorophosphate, 2-(1-Oxy-pyridin-2-yl)-1,1,3,3-tetramethylisothiouronium tetrafluoroborate, Tetramethylfluoroformamidinium hexafluorophosphate, N-Ethoxycarbonyl-2-ethoxy-1,2-dihydroquinoline, 2-Propanephosphonic acid anhydride, 4-(4,6-Dimethoxy-1,3,5-triazin-2-yl)-4-methylmorpholinium salts, (bis-Trichloromethylcarbonate, 1,1′-Carbonyldiimidazole.

33. A composition comprising a biodegradable IPN hydrogel comprising a first polymer network and a second polymer network, wherein the first polymer network comprises polyacrylamide and a biodegradable covalent crosslinker, and the second polymer network comprises an alginate polymer.

34. A composition comprising a biodegradable adhesive material comprising
a) a biodegradable IPN hydrogel comprising a first polymer network and a second polymer network, wherein the first polymer network comprises polyacrylamide and a biodegradable covalent crosslinker, and the second polymer network comprises an alginate polymer;
b) an adhesive bridging polymer comprising chitosan; and
c) a coupling agent comprising EDC and sulfated NHS.

35. The composition of claim 33 or claim 34, wherein the biodegradable covalent crosslinker is selected from the group consisting of poly(ethylene glycol) diacrylate (PEGDA 250), poloxamer diacrylate (Polox DA), gelatin methacrylate (GelMA), oxidized alginate methacrylate (OxAlgMA), hyaluronic acid methacrylate (HAMA), bis(2-methacryloyl)oxyethyl disulfide (Bis), and N,N′-bis(acryloyl)cystamine (Cys).

36. A method of making a biodegradable IPN hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks, the method comprising
mixing a first polymer and a second polymer; and
contacting the mixture with a biodegradable covalent crosslinker and an ionic crosslinker thereby making an IPN hydrogel.

37. The method of claim 36, wherein the biodegradable covalent crosslinker is selected from the group consisting of poly(ethylene glycol) diacrylate (PEGDA 250), poloxamer diacrylate (Polox DA), gelatin methacrylate (GelMA), oxidized alginate methacrylate (OxAlgMA), hyaluronic acid methacrylate (HAMA), bis(2-methacryloyl)oxyethyl disulfide (Bis), and N,N′-bis(acryloyl)cystamine (Cys) and wherein the ionic crosslinker comprises CaSO₄.

38. The method of claim 37, wherein the alginate is oxidized alginate.

39. A method of adhering a biodegradable hydrogel to a surface, the method comprising the steps of:
a) applying a solution comprising a high density primary amine polymer and a coupling agent to the hydrogel; and
b) placing the hydrogel on the surface;
wherein the hydrogel comprises a first polymer network and a second polymer network, wherein the first polymer network comprises a first polymer covalently crosslinked with a biodegradable covalent crosslinker and the second polymer network comprises a second polymer crosslinked with ionic or physical crosslinks.

40. The method of claim 39, wherein the surface is a tissue surface that is wet, dynamic, or both.

41. The method of claim 40, wherein the surface is a medical device.

42. A method of delivering a therapeutically active agent to a subject, the method comprising:
a) applying a solution comprising a high density primary amine polymer and a coupling agent to a hydrogel; and
b) placing the hydrogel on a surface in the subject;
wherein the hydrogel comprises a first polymer network and a second polymer network, wherein the first polymer network comprises a first polymer covalently crosslinked with a biodegradable covalent crosslinker and the second polymer network comprises a second polymer crosslinked with ionic or physical crosslinks, and wherein at least one therapeutically active agent is encapsulated in, or attached to the surface of, the hydrogel and/or high density primary amine polymer, thereby delivering a therapeutically active agent to the subject.

43. A biodegradable adhesive material comprising a) a hydrogel comprising a first polymer network and a second polymer network, wherein the first polymer network comprises a first polymer covalently crosslinked with a biodegradable covalent crosslinker and the second polymer network comprises a second polymer crosslinked with ionic or physical crosslinks; b) a high density primary amine polymer; and c) a coupling agent, wherein the high density primary amine polymer and the coupling agent are applied to one side of the hydrogel.

44. The biodegradable adhesive material of claim 43, wherein the material is in the form of a preformed patch or an injectable gel.

45. The biodegradable adhesive material of claim 43 or claim 44, wherein the first polymer network is modified with two reactive moieties, wherein the reactive moieties are each independently selected from the group consisting of methacrylate, acrylate, acrylamide, methacrylamide, thiol, hydrazine, tetrazine, norbornene, transcyclooctene and cyclooctyne.

46. The biodegradable adhesive material of any one of claims 43 to 45, wherein the first polymer network comprises polyethylene glycol (PEG) modified with norbornene and polyethylene glycol (PEG) modified with tetrazine.

47. The biodegradable adhesive material of claim 45, wherein the two reactive moieties react in the presence of Ca²⁺.

48. The biodegradable adhesive material of claim 45, wherein the two reactive moieties react in the presence of UV light.

49. The composition of any one of claims 43 to 48, wherein the biodegradable covalent crosslinker is selected from the group consisting of poly(ethylene glycol) diacrylate (PEGDA 250), poloxamer diacrylate (Polox DA), gelatin methacrylate (GelMA), oxidized alginate methacrylate (OxAlgMA), hyaluronic acid methacrylate (HAMA), bis(2-methacryloyl)oxyethyl disulfide (Bis), and N,N′-bis(acryloyl)cystamine (Cys).

50. The biodegradable adhesive material of any one of claims 43 to 49, wherein the alginate is oxidized alginate.
